(19) United States
(12) Patent Application Publication
(10) Pub. No.: US 2004/0053355 A1
(43) Pub. Date: Mar. 18, 2004

(54) Modular approach to on-line synthesis, drug discovery and biochemical transformations using immobilized enzyme reactors

(76) Inventors: Irving Wainer, Washington, DC (US); Nektaria Markoglou, Montreal (CA)

Correspondence Address: Thomas M Boyce, Fulbright & Jaworski, Suite2400, 600 Congress Avenue, Austin, TX, 78701, US

(21) Appl. No.: 10/296,743

(22) PCT Filed: May 28, 2001
(86) PCT No.: PCT/CA01/00777

(30) Foreign Application Priority Data
May 26, 2000 (CA) 2,309,510

(51) Int. Cl. C12P 1/00

(52) U.S. Cl. 435/41

(57) ABSTRACT

A coupled system using extremely different enzymes with incompatible cofactors and reaction conditions has been constructed using standard liquid chromatographic formats and open tubular formats. One of the significant aspects of the present invention lies in the development of the liquid chromatographic on-line enzyme cascade. This has been illustrated by the biosynthetic pathway involving dopamine beta-hydroxylase and phenylethanolamine N-methyltransferase which encompass the synthesis of the key neurotransmitters, norepinephrine and epinephrine. The results demonstrate for the first time the immobilization of dopamine beta-hydroxylase and phenylethanolamine N-methyltransferase. The IMERs are active and can be used in a liquid chromatographic format for qualitative and quantitative determinations. The IMER-HPLC system can be used to carry out standard Michaelis-Menten enzyme kinetic studies and to quantitatively determine enzyme kinetic constants, identify specific enzyme inhibitors, provide information regarding the mode of inhibition and the inhibitor constants (Kᵢ). A second significant aspect of the present invention lies in the ability of the immobilized enzyme reactors to be used independently or as a combination, thus providing a unique opportunity to explore the interrelationships between these enzymes, to investigate the source of diseases and to design new drug entities for identified clinical syndromes.

### FIELD OF THE INVENTION

The present invention relates to immobilized enzyme reactors (IMERs). More specifically, the present invention is concerned with the application of a liquid chromatographic system based upon coupled on-line immobilized enzyme reactors (HPLC-IMERs) in organic synthesis, biochemistry and pharmacology. The novel coupled enzyme system of the present invention allows for on-line chromatographic purification and structural identification of products. In an alternative embodiment, open tubular columns coupled to a mass spectrometer or other detection device may be used. Additionally, the coupled enzyme system of the present invention may be used in basic research into synthetic and metabolic pathways as well as in the discovery of new pharmaceutical substances.

### BACKGROUND OF THE INVENTION

The therapeutic and toxic effects of drugs are governed by the interactions of these molecules with biopolymers such as proteins, receptors and enzymes (Katzung, 1995). The biopolymer-drug interactions define a drug's pharmacological fate. As such, there have been interdisciplinary efforts amongst fields such as medicine, pharmacology and biochemistry to develop methods for the identification and characterization of these interactions.

In recent years there have been significant developments in the study of the basis of enzyme-drug interactions. The understanding of how enzymes react with drugs and bring about chemical changes in vivo is a key factor for the determination of drug pharmacodynamics and pharmacokinetics, and is also important in the development of new therapeutic agents. The conventional uses of enzymes within many fields have been based upon enzymes in their soluble forms (Dixon et al., 1979). In vivo most enzymes are naturally found within a cellular matrix and can be membrane bound. Consequently, many previously reported in vitro assays that utilize solubilized enzymes are not a true reflection of what is occurring in vivo. This has naturally led to the development of immobilized enzymes.

Although many methods for assaying enzymes have been developed, High Performance Liquid Chromatography (HPLC) has become an increasingly popular method of choice due to its ability to accommodate the need for increased sensitivity and versatility (Lough et al., 1995). In particular, biochromatography is a unique method in that it utilizes immobilized biopolymers and HPLC techniques in order to study drug-biopolymer interactions. It has been shown that on-line chromatography applying an immobilized enzyme reactor coupled to an analytical column allows for an ideal reflection of biological processes and quantitation of enzyme/substrate interactions (Alebic-Kolbah et al., 1993).

Many research groups have demonstrated uses of immobilized enzyme reactors to monitor and characterize metabolic and biosynthetic processes. The focus of the present invention is on the two catecholamine-system enzymes, dopamine beta-hydroxylase and phenylethanolamine N-methyltransferase and the importance of developing IMERs based upon these enzymes in order to characterize and follow the synthesis of catecholamines.

Catecholamines

Catecholamines are naturally occurring compounds that act as both hormones and neurotransmitters. The principal catecholamines are dopamine (DA), norepinephrine (NE) and epinephrine (EP). They are highly polar compounds that resemble each other chemically by the presence of an amino and a catechol group. These compounds possess a wide range of biological activities with vital roles in numerous physiological processes. The differences in their activities are attributed to the physico-chemical properties of their side chains (Schusler-Van Hees et al., 1980).

Catecholamines are located in cells in the adrenal medulla and in the central and sympathetic nervous system localized in distinct regions of the brain and ganglion (Kobayashi et al., 1992). The adrenal medulla is a gland containing chromaffin cells, which are specialized cells that manufacture, store and secrete NE or EP. It is controlled by nerves in the spinal cord and releases the catecholamines directly into the bloodstream eliciting a widespread response within the body (Carmichael et al., 1985).

Based upon the wide distribution of catecholamines, researchers have demonstrated their involvement in normal functions including heart stimulation, motor control, gastric motility and blood flow (Elenkov et al., 2000; Tani et al., 1982). These compounds are also involved in the stimulation of the autonomic nervous system in preparation of the body's “fight or flight” response to colds, fatigue and shock. Numerous neurological diseases (Alzheimer's disease, Schizophrenia, Manic depressive illnesses, and Parkinson's disease) are also associated with improper catecholamine regulation (Elenkov et al., 2000; Friedman et al., 1999).

A wide range of drugs that alter catecholamine function are used in various clinical settings. Knowledge of the catecholamine mechanisms of action is of fundamental importance in the development of new therapeutic agents for various disease states. Many studies in the literature have examined the potential sites for drug intervention in catecholamine synthesis, storage, release and metabolism.

Biosynthesis

The catecholamines, DA, NE and EP share a common synthetic pathway (FIG. 1). The pathway has been extensively studied and detailed kinetic analysis, substrate specificity, and cofactor requirements of the enzymes have been determined. Antibodies against the enzymes have allowed for the determination of their localization using immunohistochemical techniques (Liposits et al., 1986). Catecholamines are synthesized from the precursor amino acid, L-tyrosine. L-tyrosine is taken up from the circulation into catecholamine secreting neurons and adrenal medullary cells by an active transport mechanism. It then undergoes a series of chemical transformations resulting in the formation of DA, NE and EP.

Tyrosine hydroxylase (TH) is the first enzyme in the pathway, which catalyzes the formation of 3,4 dihydroxyphenylalanine (DOPA), from L-tyrosine (Nagatsu et al., 1964). TH is present in the adrenal medulla, sympathetically innervated tissues and in all catecholaminergic neurons. TH is stereospecific such that the enzyme oxidizes L-tyrosine and L-phenylalanine whereas D-tyrosine does not serve as a substrate. TH requires molecular oxygen, ferrous iron atom and tetrahydropteridin as a cofactor (Almas et al., 1996). It has been shown to display a high degree of substrate specificity and catalyzes the rate-limiting step of catecholamine biosynthesis (Nagatsu, 1995). Inhibitors of TH include amino acid analogues catechol derivatives, tropolones and iron chelators (Cooper et al., 1996).

The second step in the pathway involves the decarboxylation of DOPA to dopamine. Dopa decarboxylase (DDC), which is located in the cytoplasm of cells, is responsible for dopamine synthesis. The enzyme is found in the adrenal medulla, catecholaminergic neurons and in tissues such as liver, kidney and gastrontestinal tract (Cooper et al., 1996). Relative to the other catecholamine enzymes, dopa decarboxylase is present in excess and requires pyridoxal phosphate (vitamin B6) as a cofactor. The enzyme displays broad substrate specificity. Based upon the enzyme's ability to catalyze the decarboxylation of various L-amino acids such as DOPA, histidine and tryptophan, dopa decarboxylase was appropriately renamed to L-amino acid decarboxylase (AADC) (Voltattorni et al., 1983). The corresponding D-isomers have been shown to bind the active site and inhibit the decarboxylation (Voltattorni et al., 1983).

Some therapeutic regimens have proven successful due to the pharmacological intervention at the level of AADC. For instance, the slow degeneration of dopaminergic neurons results in the movement disorder associated with Parkinson's disease. The clinical features of this disorder are alleviated and dopaminergic activity restored by the administration of L-dopa and dopamine agonists (Katzung, 1995).

NE is synthesized in the vesicles and granules by dopamine β-hydroxylase.(DBH) and then released by exocytosis. In nerve cells, NE is released during nerve stimulation. NE is then methylated by phenylethanolamine N-methyltransferase, PNMT, the final enzyme in the pathway. What follows focuses on the final two enzymes in the pathway, dopamine β-hydroxylase (DBH) and phenylethanolamine N-methyltransferase (PNMT).

Dopamine β-hydroxylase

Dopamine β-hydroxylase (DBH) catalyzes the third step in the catecholamine biosynthetic pathway. DBH is a copper-containing protein present in higher eukaryotes and is responsible for the production of NE. DA, which is achiral, is converted to R-NE. L-NE is also designated R-NE using Cahn-Ingold-Prelog configurations. The enzyme's stereospecificity is demonstrated through the removal of the pro-R benzylic hydrogen (DeWolf et al., 1989; Wimalasena et al., 1999) (FIG. 2).

DBH is a mixed function oxidase requiring ascorbic acid and molecular oxygen for activity. It reduces one oxygen atom to water and inserts another into its substrate (Robertson et al., 1990). The enzyme is situated within catecholamine-containing granules and vesicles in contrast to the other enzymes, which are present in the cytoplasm (Wong et al., 1990; Wimalasena et al., 1991). DBH activity is highest in the adrenal medulla and in tissues such as the heart and the spleen reflecting peripheral sympathetic activity. The enzyme also appears in cerebrospinal fluid.

Dopamine and ascorbic acid are required in vivo for activity. The enzyme however does not display a high degree of substrate specificity, converting any phenylethylamine to the corresponding phenylethanolamine in vitro (Creveling et al., 1962; Farrington et al., 1990). Although ascorbic acid is the physiological reductant of the reaction, other reducing agents such as ferrocyanide and N-substituted phenylenediamines have served as ideal reducing agents in many assays. Benzylhydrazine, benzyloxyamine, and a variety of chelating agents such as tropolone and disulfiram are known inhibitors of DBH. Assaying DBH activity in tissues proves to be difficult at times due to the presence of endogenous inhibitors in the enzyme preparation (Molinoff et al., 1971). Inhibition of DBH by sulfihydryl compounds such as cysteine and glutathione is due to the chelation of the copper atom located in the enzyme's active site (Nagatsu, 1967). The effect of these inhibitors can be minimized by the addition of sulfhydryl reagents such as N-ethylmaleimide to assay mixtures. Numerous assays have been reported for assessing DBH activity. Extensive studies have been carried out in order to describe the physical and chemical characteristics of DBH.

Structure of DBH

DBH is a copper-dependent glycoprotein with a molecular weight of 290 kDa (Ishii et al., 1991). The enzyme consists of four subunits. Disulfide bonds join two of the monomers and the resulting dimers are noncovalently attached to one another to form a tetramer Robertson et al., 1994). Analyses of disulfide bonds in bovine DBH have revealed fourteen cysteine residues and seven disulfides per monomer. Molecular mass spectrometric studies have confirmed one intermolecular and six intramolecular disulfide linkages for each monomer (Robertson et al., 1994). Five of the intramolecular disulfide linkages display high densities of histidine residues. Numerous studies have been carried out to investigate the four subunits of DBH. Molecular weights ranging from 66 to 77 kDa have been reported for the individual subunits (Wong et al., 1990; Sabban et al., 1983). Studies on bovine adrenal medullary DBH demonstrate the presence of three subunits: alpha, beta and gamma that combine in a 1:2:1 ratio to form the tetramer.

DBH exists in two forms: a membrane-bound form (mDBH) that is reinternalized upon exocytosis and a soluble form (sDBH) that is stored in the granule and secreted (Ledbetter et al., 1981; Helle et al., 1984). Both forms of the enzyme have been purified and the human, bovine, rat and mouse genes have been cloned (Kobayashi et al., 1989; Wang et al., 1990; McMahon et al., 1990; Nakano et al., 1992). The two forms display differences in pH stabilities and substrate affinities. Amino acid analysis of bovine and human DBH displays high portions of glutamic acid, aspartic acid, asparginase, glycine and leucine (Lamoureux et al., 1987; Wong et al., 1990). Sequence analysis of human DBH has revealed four consensus sequences for glycosylation (Robertson et al., 1990; Wong et al., 1990). mDBH is ampiphilic and sDBH is hydrophilic in nature. MDBH, in contrast to sDBH, consists of a portion of hydrophobic amino acids which act as an anchor in the chromaffin granule. The anchor traverses the membrane with the remainder of the enzyme located in the outer surface of the membrane (Blakeborough et al., 1981).

Disorders Associated with DBH

A novel approach used to study the clinical significance of DBH has been through the use of genetically altered mice. Targeted disruption of the DBH gene has revealed the various roles the enzyme possesses in vivo. Thomas and Palmiter have shown the importance of DBH in reproduction (Thomas and Palmiter, 1998). Their investigations revealed that most of the DBH −/− mice died in utero (Thomas et al., 1995). The small proportion that survived was due to the presence of maternal catecholamines that can cross the placenta. DBH was also shown to be essential in the developmental stages as well as in the retention of certain behaviors (agatsu and Stjame, 1998). For instance, changes in maternal behavior have been shown to be due to DBH deficiency. DBH-deficient females abandon their litters whereas normal females retrieve their pups. Administration of dihydroxyphenylserine (DOPS), a DBH-independent precursor in the mothers' drinking water prior to and the day after birth resulted in the mothers' acceptance of her pups (Thomas and Palmiter, 1998).

NE is also important in energy balance, thermoregulation, immune regulation, and cardiovascular control (Alaniz et al., 1999; Thomas et al., 1997). DBH −/− mice were more susceptible to infections compared to normal mice (Alaniz et al., 1999). When mice were housed in pathogen-free environments they appeared normal. However, upon infection with Mycobacterium tuberculosis the DBH −/− mice displayed impaired T-cell function and became susceptible to infection (Alaniz et al., 1999). Similarly, NE involvement in cold acclimatization was demonstrated with the mutants' inability to adapt to cold temperatures (Thomas and Palmiter, 1998).

DBH deficiency results in a decrease of NE and EP levels and an accumulation of DA and L-dopa in urine, plasma and CSF. Changes in DBH activity are also reflected in metabolite changes. HVA and 3-methoxytyramine displays increased levels whereas VMA decreases with decreased levels of NE. DBH deficiency is diagnosed by measurement of NE/DA ratio. Normal individuals display a ten-fold difference from patients with DBH deficiency. Patients with pheochromocytoma have displayed increased blood levels of DBH (Nagatsu, 1986). Interestingly upon removal of the tumor the DBH levels are shown to decrease. The main biochemical markers for Parkinson's disease have been decreases in TH and tetrahydrobiopterin concentrations however DBH has also been show to decrease in these patients (Nagatsu et al., 1983; Hurst et al., 1985).

Additional disorders that have been identified and correlated with DBH deficiency include: spontaneous abortions, hypoglycemia, hypotension, ptosis of eyelids and occasional syncope (Robertson et al., 1991). Appropriate treatment of DBH deficiency has been challenging. Treatments utilizing compounds such as phenylpropanolamine, tranylcyprornine and metyrosine gave rise to adverse effects including paranoid thinking and increases in blood pressure reaching levels characteristic of hypertension (Robertson et al., 1991). Extensive understanding of NE pharmacology and DBH activity should allow for effective therapeutic intervention in many of the above mentioned disorders.

Phenylethanolamine N-methyltransferase

The terminal enzyme in the catecholamine biosynthetic pathway is phenylethanolamine N-methyltransferase (PNMT). PNMT catalyzes the transmethylation of NE requiring S-adenosyl-L-methionine (SAM) as a methyl donor (Pendleton and Snow, 1973). The reaction results in the production of epinephrine (EP) and S-adenosyl-L-homocysteine (SAH) (Grunewald et al., 1996) (FIG. 3). The enzyme is primarily situated in chromaffin cells of the adrenal medulla as well as in discrete regions of the brain (Park et al., 1986). Depending upon its location, EP is synthesized to function as either a hormone or as a neurotransmitter. PNMT activity has been reported in the adrenal medulla of a rabbit, rat, monkey, cow, pig, frog, mouse, dog and snake (Park et al., 1986).

Its substrate specificity has been extensively investigated, and the enzyme has been shown to display poor substrate specificity. However, the enzyme requires that a hydroxyl group at the β position of the ethylene side chain be present on the substrate for catalysis to occur (Grunewald et al., 1996). Commonly used phenylethanolamine derivatives for assaying PNMT include norepinehrine, normetanephrine, synephrine, octopamine, metanephrine and epinephrine. PNMT is inhibited by its own substrates and products in vitro (Borchardt et al., 1976). Phenylethylamines, benzylamines and a variety of sulfhydryl reagents such as p-chloromercuribenzoic acid and mercury are known inhibitors of PNMT. Assaying PNMT activity proves to be difficult due to the presence of endogenous inhibitors in the preparations. The effect of these inhibitors is overcome with MAO inhibitors such as pargyline (Molinoff et al., 1969). Radiochemical and HPLC methods are the two methods of choice to assay PNMT in various biological matrices (Lee et al., 1985). The physical and chemical characteristics of PNMT have been elucidated utilizing these and many other techniques.

Structure of PNMT

PNMT has been isolated and purified from various species and molecular weights ranging from 30 to 40 kDa have been reported (Connett and Kirshner, 1970; Park et al., 1982, Kaneda et al., 1998). It is a monomeric protein that has similar features to other N-methyltransferases such as nicotinamide N-methyltransferase (Kaneda et al., 1998). PNMT from different species have been shown to differ in charge and among some species there are multiple forms of the enzmye (Park et al., 1982; Joh and Goldstein, 1973).

Mechanism of Action

PNMT converts NE to EP through the use of SAM as a methyl donor. This reaction is one of several in the body that utilizes SAM. Other important methylation reactions in vivo include the methylation of DNA, conversion of guanidinoacetate to creatine and conversion of acetylserotonin to melatonin (Hoffman, 1984; Itoh, 1997; Jenne, 1997). Methionine is a dietary source of methyl groups. The adenosyl group of ATP is transferred to the methionine sulfur group resulting in the formation of SAM. Methyl groups attached to the sulfur group of SAM can be transferred to a nitrogen, oxygen or carbon atom of an acceptor molecule that yields the methylated product and SAH. SAH is then hydrolyzed to adenosine and homocysteine.

The PNMT reaction is believed to proceed through ordered sequential binding (Grunewald et al., 1996); SAM binding is then followed by NE (Pendleton et al., 1973). Quantitative structure-activity relationship studies have proven useful in elucidating the required conformation of the aminoethyl side chain of typical substrates of PNMT (Grunewald et al., 1988). They have indicated the presence of a compact hydrophilic pocket within the aromatic ring-binding region of the enzyme's active site (Sall and Grunewald, 1987). A coplanar relationship exists between the amine nitrogen, the aromatic ring and the active site. Once epinephrine is formed, it returns to the chromaffin granule for storage (Burke et al., 1983). The cells release EP into the bloodstream where it is capable of acting on the liver, skeletal muscle and adipose tissue.

Disorders Associated with PNMT

Hypertension is a polygenic disease. PNMT plays a role in blood pressure homeostasis (Reis et al., 1988). The ability of PNMT inhibitors to lower blood pressure in spontaneously hypertensive rats has been widely investigated (Saavedra, 1988). PNMT inhibitors reduce central epinephrine levels however they are non-selective and demonstrate α₂-adrenoceptor binding affinity (Toomey et al., 1981). The effects of the PNMT inhibitors investigated have proven to be ambiguous based upon their display of α₂-adrenoceptor affinity. Pheochromocytomas are common in the adrenal medulla These benign tumors induce an increase in the secretion of epinephrine. This results in increases in the heart rate, headaches, palpitations and weight loss. Therefore, the importance in developing potent and selective PNMT inhibitors will prove vital in the effective intervention of these disorders.

Immobilized Enzymes

Enzymes are complex proteins that are involved in a variety of chemical transformations. These molecules accelerate chemical reactions within living cells through a process that involves the formation of enzyme-substrate complexes. These complexes lower the kinetic and energetic barriers associated with a chemical transformation and result in product formation.

In the body, enzymes mediate a variety of processes ranging from digestion to synthesis to degradation. The biological importance of enzymes and their wide utility have made them primary targets for the medical, industrial and analytical fields. Indeed, there have been numerous advances in the isolation, production and purification of enzymes, which have resulted in the development of the field of enzyme technology.

In the past the conventional uses of enzymes within various scientific fields were based upon enzymes in their soluble forms (Katchalski-Katzir, 1993). Even with the enzymes' high efficiency and specificity there are numerous disadvantages and limitations. Enzymes can be costly, unstable, and difficult to recover from reactions and some are only available in minute amounts. Since enzymes are not altered during the reactions they catalyze it would be beneficial if they could be reused. With the recent advances in biotechnology there has been an increased interest in the development of immobilized enzymes (Hoffman, 1990; Liang et al., 2000; Turner et al., 1987). Immobilization stabilizes enzymes by restricting their movement and allowing for their reuse.

### OBJECTS OF TEE INVENTION

An object of the present invention is therefore to provide a liquid chromatographic system based upon coupled on-line immobilized enzyme reactors (IMERs) or, alternatively, coupled open tubular columns containing immobilized enzymes that are suitable for organic synthesis, either of which can be connected to a mass spectrometer or other detection device.

A further object of the present invention is to provide a novel coupled enzyme system that may be used in a number of biochemical and pharmacological applications, such as the on-line determination of kinetic parameters of biochemical transformations, purification and structural identification of products and the rapid identification of new pharmaceutical substances, such as inhibitors.

### SUMMARY OF THE INVENTION

The use of immobilized enzymes has steadily increased in recent years. Based upon the advantages that immobilized enzymes possess over soluble enzymes, numerous applications have emerged in biomedical and analytical fields. The present invention demonstrates the applicability of a liquid chromatographic system based on coupled on-line immobilized enzyme reactors (IMERs) to organic synthesis, biochemistry and pharmacology. The invention allows customization of systems and modular design wherein wherein chemists can add or remove the IMERs necessary for their particular synthetic goal. The system allows for on-line chromatographic purification and structural identification of products and could greatly reduce time required to identify new synthetic transformations. In addition, the construction of a coupled enzyme system provides a novel approach to basic research into synthetic and metabolic pathways as well as a rapid method for the discovery of new pharmaceutical substances.

The invention provides the construction of a coupled system using vastly different enzymes with incompatible cofactors and reaction conditions. The novelty of the invention resides both in the development of a liquid chromatographic on-line enzyme cascade and also in the demonstration and simulation of a biologically relevant catecholamine biosynthetic pathway. The biosynthetic pathway involving dopamine beta-hydroxylase and phenylethanolamine N-methyltransferase comprise the synthesis of the key transmitters, norepinephrine and epinephrine. The invention demonstrates the immobilization of dopamine beta-hydroxylase and phenylethanolamine N-methyltransferase. The IMERs are active and can be used in a liquid chromatographic format for qualitative and quantitative determinations. Previous studies with IMER-HPLC systems have shown that the activity of the immobilized enzymes reflects the non-immobilized enzymes. Thus, the IMER-HPLC system can be used to carry out standard Michaelis-Menten enzyme kinetic studies and to quantitatively determine enzyme kinetic constants, identify specific enzyme inhibitors, provide information regarding the mode of inhibition and the inhibitor constants (Kᵢ). The immobilized enzyme reactors of this invention, used independently or in combination provide a unique opportunity to explore the interrelationships between these enzymes, to investigate the source of catecholamine-related disorders and serve as rapid throughput screening tools to design new drug entities as either substrates or inhibitors for identified clinical syndromes.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

FIG. 1 Pathway of the biosynthesis of catecholamines.

FIG. 2 Stereospecific reaction of dopamine beta-hydroxylase.

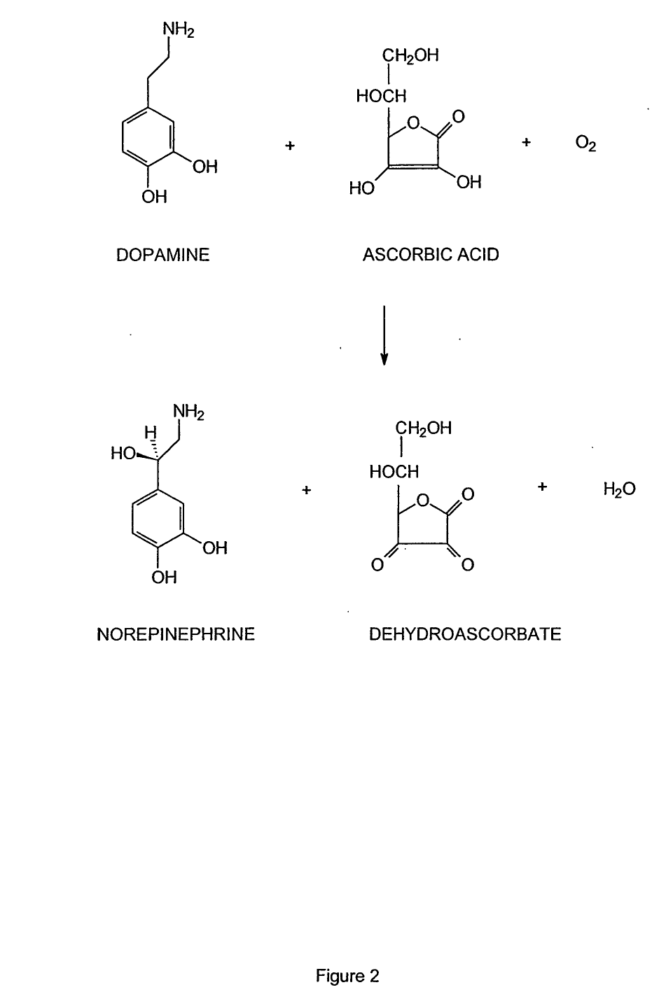

FIG. 3 The final step in the biosynthesis of epinephrine.

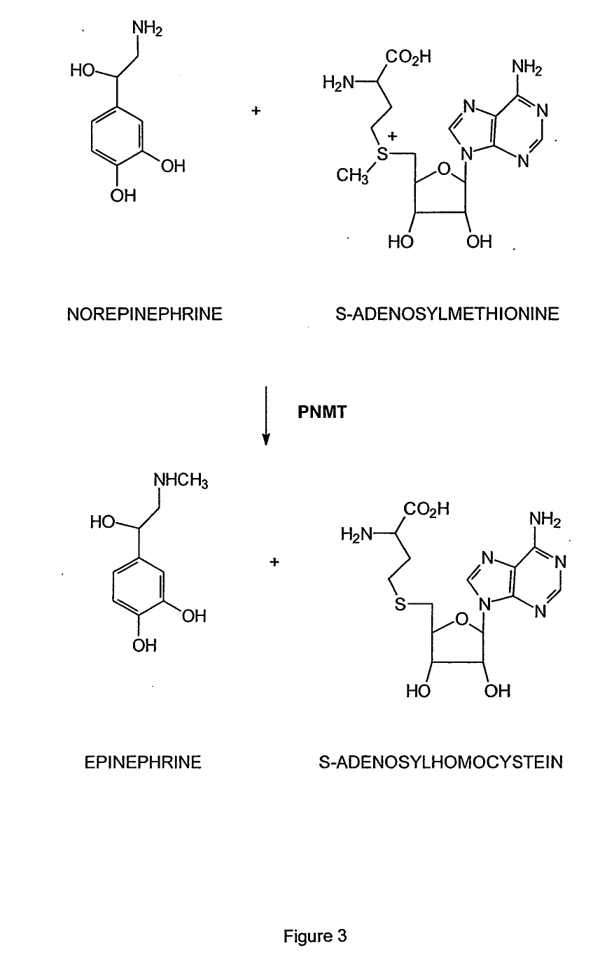

FIG. 4 Representative chromatograms from DBH assays analyzed: A: Blank reaction mixture containing a boiled enzyme; B: Reaction mixture; C: Reaction mixture under chiral conditions (Crownpak CR(+) column).

FIG. 5 Representation of DBH immobilized onto two different supports in order to mimic mDBH and sDBH. A: DBH-IAM; B: DBH-Glut-P.

FIG. 6 Schematic representation of on-line DBH IMER HPLC system.

FIG. 7 Representative chromatograms of on-line hydroxylation of tyramine A:control on DBH-Glut-P IMER B: reaction on DBH-Glut-P IMER C: reaction on DBH-IAM IMER

FIG. 8 Inhibition of PNMT activity of both PNMT and PNMT-SP as a function of benzylamine.

FIG. 9 Schematic representation of on-line PNMT-IMER HPLC system.

FIG. 10 Representative chromatograms of on-line N-methylation of normetanephrine. A: Injection of NM/SAM mixture; B: Injection of NM only.

FIG. 11 Representative chromatogram of coupled IMER system.

FIG. 12 Open tubular, PNMT immobilized, reaction mixture (substrate with cofactor)

FIG. 13 Open tubular, PNMT immobilized, control solution (substrate)

### DESCRIPTION OF THE PREFERRED EMBODIMENTS

List of Abbreviations

| | |
|---|---|
| AADC | Aromatic amino acid decarboxylase |
| ACHT | α-chymotrypsin |
| ATP | Adenosine triphosphate |
| CN | Cyano |
| COMT | Catechol-O-methyltransferase |
| DA | Dopamine |
| DBH | Dopamine beta-hydroxylase |
| DDC | Dopa-decarboxylase |
| DOPA | dihydroxyphenylalanine |
| E | enzyme |
| EP | Epinephrine |
| ES | Enzyme-substrate complex |
| Glut-P | Glutaraldehyde-P |
| HLADH | Horse liver alcohol dehydrogenase |
| HPLC | High performance liquid chromatography |
| HVA | Homovanillic acid |
| IAM | Immobilized Artificial Membrane |
| IMER | Immobilized Enzyme Reactor |
| Kᵢ | Inhibitor constant |
| Kₘ | Michaelis constant |
| M | Metanephrine |
| MAO | Monamine oxidase |
| mDBH | membrane bound dopamine beta-hydroxylase |
| NE | Norepinephrine |
| NM | Normetanephrine |
| OCT | Octopamine |
| ODS | Octadecyl |
| P | Product |
| PNMT | Phenylethanolamine N-methyltransferase |
| S | Substrate |
| SAM | S-adenosyl-L-methionine |
| SAH | S-adenosyl-L-homocysteine |
| sDBH | soluble dopamine beta-hydroxylase |
| SP | Stationary phase |
| SYN | Synephrine |
| TH | Tyrosine Hydroxylase |
| TYR | Tyramine |
| Vₘₐₓ | maximal rate |
| VMATs | Vesicular monoamine transporters |

### EXAMPLE 1

### SYNTHESIS AND CHARACTERIZATION OF IMMOBILIZED DOPAMINE β-HYDROXYLASE IN MEMBRANE-BOUND AND SOLUBILIZED FORMATS

Introduction

Dopamine is hydroxylated to norepinephrine by the catalytic effect of dopamine beta-hydroxylase (DBH), one of the enzymes involved in catecholamine biosynthesis (Wong et al., 1990). The enzymatic process involves the oxidation of ascorbic acid to dehydroascorbate and the reduction of Cu²⁺ to Cu⁺ (Friedman et al., 1965). The enzyme is involved in the regulation of blood pressure by the nervous system and a target for antihypertensive drugs (Lewis et al., 1992). DBH is situated within catecholamine-containing chromaffin granules and exists in two forms: a membrane-bound form that is reinternalized upon exocytosis and a soluble form that is stored in the granule and secreted (Helle et al., 1984). The two states of the enzyme in the chromaffin granules are immunochemically identical, however they display differences in pH stability and substrate affinity (Nagatsu et al., 1972).

Given the key role that DBH plays in the biosynthesis of catecholamines, this enzyme has been subject of a number of studies. The majority of assays utilize crude extracts giving rise to the possibility of endogenous substances in such extracts that can interfere with DBH activity (Molinoff et al., 1971). When highly purified enzyme is used it requires long and complicated purification procedures that are costly. The enzymatic assays can also be cumbersome. For example, one assay uses a two-step enzyme method, using phenylethanolamine N-methyltransferase and S-adenosyl-L-methionine (Molinoff et al., 1971). However, with these techniques saturating substrate concentrations for DBH cannot be examined due to the inhibition of the second enzyme, phenylethanolamine N-methyltransferase.

The main problem faced by researchers when characterizing DBH is the repeated need for large amounts of highly purified enzyme. However there are recent applications that utilize enzymes in one or more immobilized form (Bickerstaff, 1997, Bickerstaff, 1984). The enzymes were immobilized onto solid supports and used in batch incubations (Bickerstaff, 1997; Lowe et al., 1990) or as biosensors (Bickerstaff, 1984; Lowe et al., 1990; Marko-Varga et al., 1994; Johansson et al., 1993; Nordling et al., 1993; Tischer et al., 1999). These applications do not require highly purified enzymes and decrease the amounts of enzyme required. The immobilized enzymes retain their activity and can be reused following a simple washing procedure.

In this invention, the hydrophobic character of the LAM stationary phase was used to immobilize commercially available partially purified dopamine beta-hydroxylase. The IAM interphase is derived from the covalent immobilization of 1-myristoyl-2-[(13-carboxyl)tridecanoyl]-sn-3-glycerophospholine on amino-propyl silica, and resembles one-half of a cellular membrane (Pidgeon et al., 1992). The hydrocarbon chains create interstitial spaces that allow for the insertion of DBH.

DBH was also immobilized onto glutaraldehyde-P (Glut-P), a wide-pore silica that has been covalently clad with polyethyleneimine, a hydrophilic polymer (Narayanan et al., 1990). The reactive amine groups of the polymer form a covalent bond with glutaraldehyde. This particular support is ideal for immobilization of proteins with primary amino groups that form an amine-aldehyde Schiff linkage with Glut-P (Narayanan et al., 1990).

The utility of the DBH-IAM phase and the DBH-Glut-P phase was investigated by determining both qualitative and quantitative aspects of enzyme kinetics, comparing both the free and immobilized enzyme. The results confirm that the DBH-IAM and DBH-Glut-P interphases retained their respective enzymatic activity.

A chiral assay was developed to demonstrate that the immobilized enzyme retained its stereospecificity. Investigations were conducted to determine the effect of pH, buffer type, substrate concentration, cofactor concentration and sensitivity to inhibitors. The results demonstrate that the IAM-immobilized and Glut-P immobilized dopamine beta-hydroxylase can be utilized to screen substrates and inhibitors of membrane-bound DBH and soluble DBH, and to provide information concerning their pharmacological properties.

Materials and Methods

Materials

Dopamine-beta-hyrdoxylase (from bovine adrenals), catalase (from bovine liver), DL-octopamine hydrochloride, tyramine hydrochloride, (±)-norepinephrine bitartrate salt, (−)-norepinephrine bitartrate salt, dopamine hydrochloride, ascorbic acid, and other chemicals unless otherwise stated were obtained from Sigma Chemical Company (St. Louis, Mo., USA). Hexane (95% n-hexane), methanol and glacial acetic acid, all HPLC grade were manufactured by J. T. Baker (Phillipsburg, N.J., USA) and purchased through Moquin Scientific (Montreal, QC, Canada). The IAM.PC (12 μm, 300 Å) non-endcapped chromatographic support was obtained from Regis Chemical Co. (Morton Grove, Ill., USA). The IAM.PC bonded phase, according to the manufacturer, contains a near monolayer of C14 saturated phosphatidylcholine, covalently linked to silica through an amide link. Glutaraldehyde-P affinity packing (40 μm, 300 Å) was obtained from J. T. Baker Inc.

Apparatus

The chromatographic experiments were carried out using a Thermo Separation Products P1000 pump (ThermoQuest, San Jose, Calif., USA) and a Thermo Separation Products AS3000 autosampler equipped with a 100 μl loop. The solutes were detected using an ABI fluorescence detector (ABI Analytical, Ramsy, N.J.). Data was collected using a Thermo Separation Products Cbromjet integrator interfaced with a Spectra 486 computer equipped with OS2 software for data collection. A 5 μm Prodigy C18 stationary phase packed in 250×4.6 mm column (Phenomenex, Torrance, Calif., USA) and a 5 μM nitrile guard cartridge (Regis Chemical Co., Morton Grove, Ill., USA) were used for the chromatography.

Chromatographic Procedures

The chromatographic separation of the different substrates and products was achieved with a mobile phase consisting of potassium phosphate buffer (25 mM) adjusted to pH 2.0 with trifluoroacetic acid. The solutes were quantitated using fluorescence detection with excitation at λ=265 nm and no cut-off filter for emission. A flow rate of 1 ml/min and ambient temperature were used throughout the study. Under these chromatographic conditions the relative chromatographic retentions (k′ values) of norepinephrine, octopamine, dopamine and tyramine are 0.68, 1.27, 4.79, and 8.86, respectively.

Examination of the enzyme's stereospecificity was performed on the same chromatographic system described above using a column containing a chiral crown ether based chiral stationary phase, Crownpak CR(+) packed in 250×4.6 mm column (Regis Chemical Co.) The mobile phase consisted of aqueous perchloric acid (0.01M, pH 2) at a flow rate of 1 ml/min and at ambient temperature. The relative chromatographic retentions (k′ values) of R-(+)-norepinephrine and S-(−)-norepinephrine were 3.98 and 4.38, respectively and the observed enantioselectivity (a) was 1.10. The solutes under chiral conditions were monitored using UV detection at λ=278 nm.

Extraction of Catecholamines

Enzymatic conversions utilizing DBH require cupric sulphate, catalase, ascorbic acid and sodium fumarate. An off-line extraction method utilizing solid phase extraction cartridges containing a phenylboronic acid stationary phase was developed. The procedure consisted of the following: 1-ml cartridges were conditioned with 2 ml of methanol followed by 2 ml of sodium phosphate buffer (0.1M, pH 8.4). One ml of reaction mixture was added, the cartridge was then washed with 1 ml sodium phosphate buffer (0.1M, pH 8.4) and the substrate and product were eluted using 1 ml of 0.1N HCl. The eluate was directly injected onto the HPLC system.

Assay for Dopamine Beta-Hydroxylase Activity

Dopamine beta-hydroxylase activity was assayed using a modified procedure derived from the work of Nagatsu (Nagatsu, 1991). The enzyme was assayed as follows: four different solutions were prepared A, B, C and D (final concentration). (A) 500 μL of enzyme solution (10 μg/ml); B: 275 μL sodium acetate buffer (10 mM, pH 5.5), 50 μL cupric sulphate (5 μM); C: 25 μL catalase (5 μg/ml), 50 μL ascorbic acid (10 mM), 50 μl sodium fumarate (10 mM); D: 50 μl substrate (5 mM). Solution B was added to solution A, the resulting solution was mixed for 1 min. Solution C was added and the resulting solution was mixed for an additional minute. The reaction was started with the addition of the substrate (D), after which the reaction solution was incubated for 5 min at 37° C. in a shaking bath. The reaction was stopped by the addition of 100 μl of cold hexane, the resulting mixture was centrifuged at 3000×g for 10 min and the supernatant transferred to the pre-conditioned phenylboronic acid cartridges in order to extract the product formed and the remaining substrate. Extracted samples were directly injected onto the HPLC under either chiral or achiral conditions.

Immobilization of DBH onto IAM

The packing material was washed three times with sodium acetate buffer (0.1M, pH 5.5). The washing was carried out by adding 1 ml of buffer to the packing material, the suspension was vortexed for 1 min, centrifuged at 3000×g for 10 min and the supernatant decanted. The enzyme solution (8-10 μg in 1 ml sodium acetate buffer) was added to the packing material, the mixture was vortexed for 15 min and then placed in a shaking bath for 2 h at ambient temperature. At the end of 2 h, the suspension was centrifuged, the supernatant decanted and the packing material washed three additional times with buffer. The amount of enzyme immobilized on the packing material was determined by measuring the amount of residual enzyme in the supernatant using the BioRad Protein Assay. The difference in the absorbance reading before immobilization and the total of absorbances after immobilization determined the amount of enzyme bound on the packing material.

Immobilization of DBH onto the Glut-P Interphase

The packing material was washed three times with sodium acetate buffer (0.1M, pH 6). The washing was carried out by adding 1 ml of buffer to the packing material, the suspension was mixed for 2 min, centrifuged at 3000×g for 10 min and the supernatant decanted. The enzyme solution (8-10 μg in 1 ml sodium acetate buffer] was added to the packing material, the mixture was vortexed for 2 min and then placed on a shaker/rotator for 6 h at ambient temperature. At the end of 6 h, the suspension was centrifuged, the supernatant decanted and the packing material washed three additional times with the buffer. The amount of residual enzyme was determined utilizing the same procedure as that carried out for the enzyme immobilized onto IAM.

Regeneration of Enzyme Activity

After each reaction, the activity of the immobilized enzyme was regenerated using a simple washing procedure. One ml of sodium acetate buffer at the appropriate pH was added to the DBH-IAM or DBH-Glut-P material, the mixture was mixed for 2 min, centrifuged for 15 min, and the supernatant was discarded. After this process was repeated an additional 3 times, the enzyme was active. The DBH-IAM phase remained active for a six-month period when stored in sodium acetate buffer (0.1M, pH 7) at 4° C. The DBH-Glut-P material was stored in sodium acetate buffer (0.1M, pH 6) at 4° C. and remained active for over three months.

Results

Chromatographic Results

Octopamine, norepinephrine, tyramine and dopamine were resolved, under the chromatographic conditions used in this study. Standard curves for the substrates and products were linear over the range investigated with regression equations and correlation coefficients: y=9E+06 x+3E+06, r=0.9777; y=1.3E+07×-9.6E+03, r=1.000; y=3E+07×-21963, r=1.000 and y=1.12E+07x-6.7E+04, r=0.9978 for dopamine, tyramine, octopamine and norepinephrine respectively. Recoveries for both the substrates and products exceeded 75%; dopamine, 94±2.1%, norepinephrine, 76±1.6%, octopamine, 84±1.1% and tyramine, 89±2.4%.

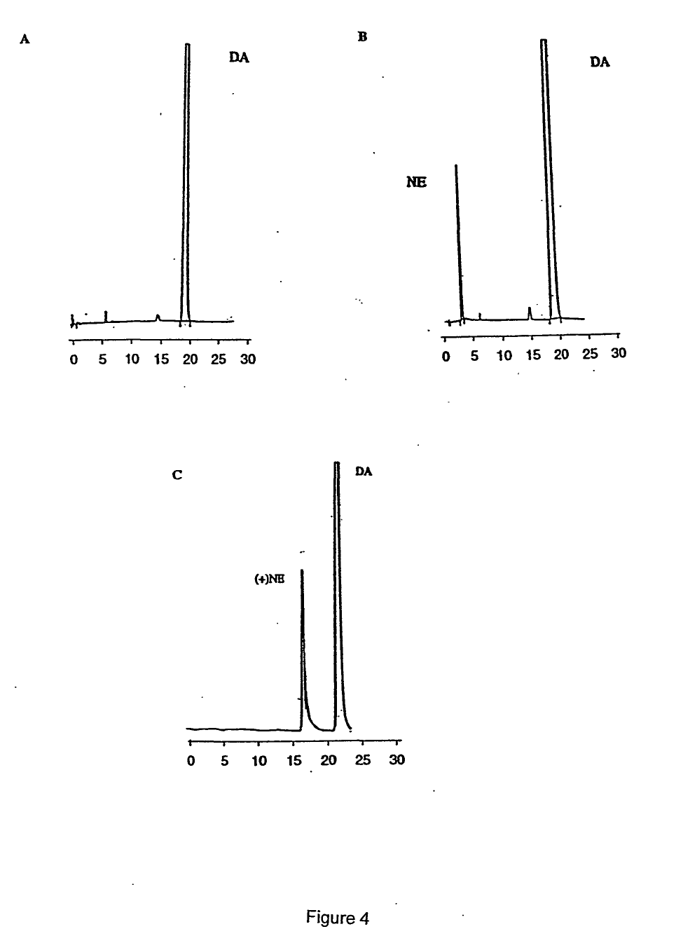

Typical chromatograms resulting from the analysis of blank reaction mixture containing boiled enzyme and reaction mixture are shown in FIGS. 4A and B. The results show that the product enzymatically formed by DBH can be isolated by HPLC and detected by fluorescence detection. A chiral HPLC assay was developed utilizing a Crownpak CR(+) chiral stationary phase in order to determine if the immobilized enzyme retains its native stereospecificity. FIG. 4C displays the formation of R-(+)-norepinephrine by immobilized dopamine beta-hyroxylase when dopamine is used as a substrate. Therefore, immobilization of DBH on the IAM stationary phase does not alter the enzyme's activity or stereospecificity. DBH was also shown to retain its stereospecificity when immobilized on the Glut-P interphase.

Optimization of DBH Immobilization

The quantity of DBH, which could be immobilized on the IAM, was investigated. DBH (10 μg in 1 ml of sodium acetate buffer (0.1M, pH 7)) was stirred with 1-10 mg of IAM. The amount of immobilized enzyme and the rate of the reaction were investigated for the different amounts of IAM packing material. The amount of immobilized enzyme was determined utilizing the BioRad assay. DBH (8.67±0.55 μg) was immobilized on 2 mg of the IAM material. When greater than 2 mg of LAM was used to immobilize 10 μg of enzyme, over 85% of the enzyme was immobilized. However, it was found that with increasing amounts of IAM there was a decrease in the rate of reaction. Under the experimental conditions, the enzyme and substrate concentrations were held constant with increasing amounts of IAM.

The amount of DBH immobilized on the Glut-P interphase was also investigated. DBH (10 μg in 1 ml of sodium acetate buffer (0.1M, pH 6)) was mixed with different amounts of Glut-P utilizing a rotator/stirrer. When greater than 10 mg of Glut-P were used to immobilize 10 μg of DBH, over 70% of the enzyme was immobilized. Increases in the rate were visible with decreasing amounts of Glut-P. In the present experiments the optimal conditions are achieved at 10 μg of DBH being immobilized onto 50 mg of Glut-P.

Comparison of Immobilized and Non-Immobilized DBH

Effect of Buffer Composition on DBH Activity

The hydroxylation activity of free and immobilized DBH was determined by examining a series of compounds known to be substrates and products of the enzyme. The free and immobilized enzyme activities were determined by following the formation of the norepinephrine or octopamine from the β-hydroxylation of dopamine or tyramine, respectively. The effect of concentration and composition of buffer on enzymatic activity was examined. Phosphate buffer was found to be inhibitory compared to sodium acetate when dopamine and tyramine were utilized as substrates. Similarly at sodium acetate buffer concentrations exceeding 0.2M an inhibitory effect is observed for both the free and immobilized enzyme. Therefore, 0.1M sodium acetate buffer was used for the assays allowing for maximal enzyme activity.

Effect of Incubation Time and Enzyme Concentration on DBH Activity

The length of incubation and the amount of enzyme used per assay were varied independently. Optimal conditions consisted of a 5-min incubation with 10 μg of dopamine β-hydroxylase. The amount of dopamine β-hydroxylase that was prepared for each experiment was determined utilizing the BioRad assay each time. The activity of free and immobilized DBH as measured with substrates, dopamine and tyramine, is linear up to 20 μg of enzyme under the assay conditions.

Effect of pH on DBH Activity

The activity of the free and the two immobilized forms of dopamine β-hydroxylase was measured at different pHs (at constant ionic strength buffers) to determine the optimum pH for the two forms of the enzyme (DBH-IAM and DBH-Glut-P). A pH optimum of 5.5 was found for non-immobilized dopamine P-hydroxylase, which is consistent with previously reported values (Nagatsu, 1991; Cooper et al., 1996). There was a shift of 1.5 pH units for the immobilized enzyme onto IAM, pH 7. A pH optimum of 6.0 was found for the DBH-Glut-P interphase. Similar pH profiles were obtained when substrates, dopamine and tyramine were utilized. The optimum pH was utilized at optimal conditions for the non-immobilized and the two forms of immobilized DBH.

Effect of Catalase on DBH Activity

Catalase has been reported to stimulate in vitro the activity of DBH (Nagatsu, 1991). For the free enzyme it was found that with increasing concentrations of catalase there was an increase in the enzyme activity. In contrast, the enzyme immobilized onto LAM did not display increased activity with higher amounts of catalase. Similar results were obtained when sodium fumarate was examined. Therefore, unlike the free enzyme, the immobilized enzyme does not require the presence of catalase and sodium fumarate to stimulate its activity. Similar findings were obtained with the enzyme immobilized onto Glut-P.

Effect of Cupric Ions on DBH Activity

For all forms of the enzyme, optimal enzyme activity is achieved with a Cu²⁺ concentration of 5 μM. However, with increasing concentrations of cupric sulphate inhibition is observed for all forms of the enzyme (Nagatsu, 1991). This is consistent with previously reported findings.

Effect of Substrate and Cofactor Concentration on DBH Activity

The effect of tyramine and dopamine concentrations on the enzymatic activity of non-immobilized and immobilized DBH was examined. The kinetic parameters were calculated for all forms of the enzyme using standard Michaelis-Menten approach. Lineweaver-Burke plots were used to calculate the Michaelis constant (Kₘ) (Dixon and Webb, 1979). The rates of reaction (Vₘₐₓ) were calculated using μmol/mg/min for non-immobilized DBH (specific activity) and immobilized DBH (apparent-specific activity). The data was calculated using mg of immobilized or non-immobilized DBH in order to accurately compare the enzymatic activities in the two formats. The results are shown in Table 1. Parallel line patterns were obtained in double reciprocal plots with dopamine or tyramine as a varied substrate at fixed ascorbic acid concentrations. The kinetic properties of the DBH-Glut-P interphase are similar to the non-immobilized enzyme giving rise to a ping-pong mechanism. The DBH-IAM interphase does not represent a ping-pong mechanism consistent with previous reports on membrane-bound DBH (Miras-Portugal et al., 1975).

TABLE 1
Kinetic parameters (Kₘ and Vₘₐₓ) of non-immobilized and
immobilized forms of dopamine β-hydroxylase (DBH,
DBH-IAM, DBH-Glut-P).

| | Kₘ (mM) | Vₘₐₓ (μmol/mg/min) |
|---|---|---|
| Non-immobilized DBH | | |
| Tyramine | 2.85 | 0.208 |
| Dopamine | 5.03 | 0.320 |
| Ascorbic Acid | 0.62 | 0.185 |
| DBH-IAM | | |
| Tyramine | 1.64 | 0.101 |
| Dopamine | 2.53 | 0.160 |
| Ascorbic Acid | 0.52 | 0.112 |
| DBH-Glut-P | | |
| Tyramine | 1.04 | 0.112 |
| Ascorbic acid | 1.10 | 0.047 |

Effect of Temperature on DBH Activity

The non-immobilized enzyme was shown to have optimal activity at 40° C. after which the activity decreased with increasing temperature. Similar results were observed for the DBH-Glut-P interphase. The enzyme immobilized onto LAM however displayed an increase in its thermal stability. Maximal activity was maintained between 35° C. and 55° C. followed by a decrease in activity at temperatures greater than 60° C.

The Arrhenius plots for the enzyme forms were derived. The logarithm of the enzyme activity is plotted against the reciprocal of the absolute temperature (Dixon and Webb, 1979). Data yielded a linear relationship for the non-immobilized enzyme with activation energy calculated to be 20.54 kJ/mol. A straight line was also obtained for the enzyme immobilized onto the Glut-P interphase, yielding an activation energy of 38.37 kJ/mol. The Arrhenius plot for the enzyme immobilized onto IAM gave rise to a break in the curve at 37° C. An activation energy of 11.36 kJ/mol was observed above 37° C. and the activation energy below this temperature was 15.78 kJ/mol.

Effect of Inhibitor Concentration on DBH Activity

Fusaric acid is a known inhibitor of dopamine β-hydroxylase (Nagatsu et al., 1970). The effects of this compound on the enzymatic activity of non-immobilized and DBH-IAM was examined and the results are presented in Table 2. Fusaric acid was found to inhibit the DBH mediated formation of norepinephrine from dopamine at concentrations as low as 10⁻⁶M, the inhibition for both the immobilized and non-immobilized enzyme was approximately 50%.

TABLE 2
The effect of fusaric acid on the enzymatic activities
of non-immobilized dopamine (DBH) and
immobilized dopamine beta-hydroxylase onto IAM (DBH-IAM).

| | | |
|---|---|---|
| [Fusaric acid] | % Control Activity | % Control Activity |
| (nM) | DBH | DBH-IAM |

| | | |
|---|---|---|
| 0 | 100 | 100 |
| 1.6 | 99 | 91 |
| 3.2 | 84 | 73 |
| 6.3 | 40 | 56 |
| 12.5 | 21 | 51 |
| 25.0 | 19 | 46 |
| 50.0 | 17 | 20 |
| 100 | 0 | 0 |
| 200 | 0 | 0 |

Captopril is another known inhibitor of dopamine β-hydroxylase (Mueller et al., 1999). The effect of captopril on immobilized DBH kinetics is represented in the form of a reciprocal velocity plot versus the reciprocal of the concentration of tyramine. The plot indicates noncompetitive inhibition with respect to tramine. The slope and intercept replots were linear (r>0.995). Captopril was found to inhibit the DBH mediated formation of octopamine from tyramine at concentrations as low as 150 μM, the inhibition for both the immobilized and non-immobilized enzyme was approximately 50%. The results are consistent with previously reported results demonstrating that captopril is an inhibitor of DBH (Mueller et al., 1999).

Discussion

The results of this study demonstrate that the immobilized enzymes are indeed active and retain their stereospecificity. Maximal adsorption and maximal activity was achieved at 2 mg of IAM and at 50 mg Glut-P. Decreasing amounts of LAM and Glut-P at a fixed enzyme concentration gives rise to increased rate of reaction. At a fixed enzyme concentration and increasing amounts of IAM and Glut-P, accessibility of the substrate and cofactors is reduced, therefore diluting the rate. The immobilized enzymes were shown to be reusable and capable of testing different substrates after regeneration was carried out. A variety of substrates can therefore be examined once the enzyme is immobilized limiting the amount of enzyme to be used.

Many assays in the literature for DBH require the use of catalase to protect the active site from hydrogen peroxide, which is a by-product in the initial step of the reaction. For the non-immobilized enzyme there was a visible increase in the rate with increasing amounts of catalase. However, for both types of immobilized enzyme minimal amounts of catalase are required for maximal activity. The rates for both forms of immobilized enzyme (see Table 1) are lower relative to the non-immobilized DBH. This suggests the enzyme is working slower and therefore the amount of hydrogen peroxide produced as a by-product is minimal and does not affect the enzyme activity. The immobilized enzymes have conformations that do not require large amounts of catalase for protection.

Under ideal assay conditions the Kₘ and Vₘₐₓ for different substrates were determined for all the enzyme forms. In both cases, immobilization onto Glut-P and IAM, the Kₘ and Vₘₐₓ decreased. This is understandable when one considers the effects of diffusion. The diffusion of the substrate from the bulk solution to the microenvironment of the immobilized enzyme can limit the rate. This in turn affects the concentration of the substrate/cofactor in the vicinity of the enzyme. However, differences in the kinetic properties of the DBH-Glut-P and DBH-IAM interphases were observed. The DBH-Glut-P interphase similar to the non-immobilized enzyme gave rise to a ping-pong mechanism. The DBH-IAM interphase did not display a ping-pong mechanism, which is consistent with previous findings (Miras-Portugal et al., 1975). The different forms of immobilization explain these findings. When DBH is immobilized on the Glut-P interphase the enzyme is outside the stationary phase whereas with the IAM interphase the enzyme is embedded within the interphase surroundings. As such the DBH-Glut-P interphase can be utilized to characterize the enzyme found in the cytosol and information concerning the membrane-bound enzyme can be obtained with the DBH-IAM interphase.

Fusaric acid is a potent inhibitor of DBH. Inhibition of DBH has been shown to result in decreased sympathetic activity and marked hypotensive effects (Nagatsu et al., 1970). The results are consistent with previously reported results demonstraing that fusaric acid is capable of reducing endogenous levels of norepinephrine (Nagatsu et al., 1970). Fusaric acid was found to inhibit the DBH mediated formation of NE for DA at concentrations as low as 10⁻⁶ M. Fifty percent inhibition was achieved at similar concentrations for both enzyme forms.

Captopril contains a sulfhydryl moiety, which has been shown to be responsible for the attenuation of the vasoconstriction induced by sympathetic nerve stimulation (Mueller et al., 1999). Sulfhydryl compounds are known to inhibit DBH in vivo and in vitro. Noncompetitive inhibition with respect to tyramine is observed at concentrations as low as 150 μM. The inhibitory effect of captopril was shown to be reversed in a dose-dependent manner by cupric ions. The reversal of the inhibition was achieved with 2.5 μM cupric sulphate. Palatini et al showed reversal of the inhibition by 140 μM captopril at 1.5 μM Cu²⁺, as CUSO₄(Palatini et al., 1989).

DBH in chromaffin granules of the adrenal medulla occurs in a soluble form and a membrane bound form. The amino acid compositions of these two states of DBH are essentially identical (Aunis et al., 1977). However the two states have shown differences in pH stabilities. The non-immobilized enzyme was shown to have a pH optimum at pH 6.0. Consistent with our results the DBH-LAM interphase displayed a shift of 1.5 pH units, pH 7. The observed optimum pH for immobilized DBH mimics physiological conditions for the membrane bound DBH. The support utilized for immobilization, LAM, mimics the membrane environment that membrane bound DBH is accustomed to. The LAM support contains covalently bound phospholipids which is an ideal support due to the fact that the membrane bound DBH has been shown to be linked with lipids of the membrane (Pidgeon et al., 1992).

In a study carried out by Aunis et al. the properties of soluble DBH and membrane bound were examined (Aunis et al., 1977). The membrane bound enzyme was shown in contrast to the soluble form to have thermal denaturation at higher temperatures of 43.5-44° C. Similar discontinuities in the Arrhenius plots were obtained for the membrane bound DBH by the authors. The findings herein demonstrate that the DBH-IAM and DBH-Glut-P interphases are representative of the membrane-bound and soluble enzyme.

Based on the present results, the immobilized DBH interphases have now been integrated into a flow system. In-line immobilized reactors based upon the two interphases have been developed and assembled onto an HPLC analytical system. The HPLC system is used for the generation, separation and identification of substrates as well as for the identification of inhibitors of enzymatic activity. The assembly is ideally suited to screen substances for their pharmacological properties toward membrane bound and soluble forms of DBH.

### EXAMPLE 2

### ON-LINE SYNTHESIS UTILIZING IMMOBILIZED ENZYME REACTORS BASED UPON IMMOBILIZED DOPAMINE BETA-HYDROXYLASE IN MEMBRANE-BOUND AND SOLUBILIZED FORMATS

Introduction

Dopamine is converted to norepinephrine by dopamine beta-hydroxylase (DBH). DBH is a key enzyme involved in the regulation of blood pressure by the nervous system and a target for antihypertensive drugs (Lewis et al., 1992). Compounds such as benylhydrazines, benzyloxyamines as well as substances acting by copper chelation such as tropolone are known inhibitors in vivo and in vitro of DBH (Boulton, 1990). DBH is a copper containing protein and does not show a high degree of substrate specificity oxidizing a variety of substrates. The resultant structurally analogous metabolites are capable of replacing norepinephrine at noradrenergc nerve endings therefore functioning as “false neurotransmitters” (Grunewald et al., 1996).

The enzyme is situated within catecholamine-containing chromaffin granules in contrast to the other catecholamine-synthesizing enzymes (tyrosine-hydroxylase, dopa decarboxylase and phenylethanolamine N-methyltransferase) that are present in the cytoplasm (Boulton., 1990). DBH exists in two forms, the membrane bound (mDBH) which is reinternalized upon exocytosis and the soluble form (sDBH) which is stored in the granule and secreted (Grunewald et al., 1996). sDBH and MDBH are composed of four subunits (Richard et al., 1988) and display differences in pH stability and substrate affinity (Nagatsu et al., 1972).

The majority of the information obtained concerning DBH has been through enzymatic assays that have utilized the soluble form of the enzyme. Since the two forms have distinct differences it is important to carryout assays that investigate the effects of substrates and inhibitors on both enzyme forms. We have previously reported the immobilization of DBH onto solid supports that could be used in batch incubations to investigate the qualitative and quantitative aspects of sDBH and mDBH kinetics (Markoglou et al., 2001). The immobilized artificial membrane (LAM) and glutaraldehyde-P (Glut-P) stationary phases were used. The findings of the study demonstrated that through the use of different immobilization procedures and different supports the two enzyme forms could be characterized.

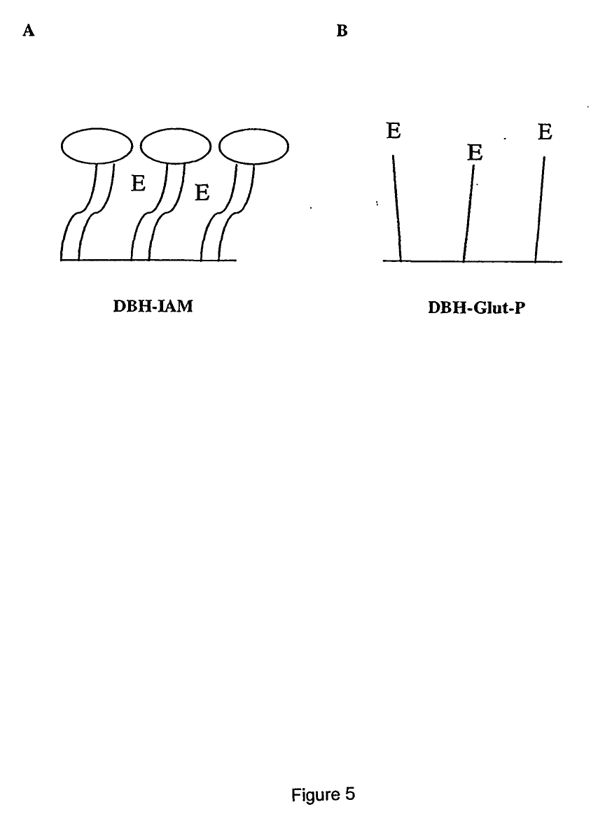

The IAM-SP is derived from the covalent immobilization of 1-myristoyl-2-[(13-carboxyl)tridecanoyl)]-sn-3-glycerophosphocholine on amino-propyl silica, and resembles one-half of a cellular membrane (Pidgeon et al., 1992). In the IAM-SP, the phosphatidylcholine headgroups form the surface of the support and the hydrocarbon side chains produce a hydrophobic interface that extends from the charged headgroup to the surface of the silica. With the IAM interphase, DBH is embedded within the interphase surroundings (FIG. 5A). The results of the study confirmed that information concerning mDBH could be obtained with the DBH-LAM interphase (Markoglou et al., 2001)

Glutaraldehyde-P is a wide pore silica that has been covalently clad with a hydrophilic polymer, polyethleneimine (Narayanan et al., 1990). Immobilization of DBH onto the interphase results in formation of an amine-aldehyde Schiff linkage with Glut-P (Markoglou et al., 2001; Narayanan et al, 1990). The DBH-Glut-P interphase proved to be useful in the characterization of the soluble form of the enzyme (FIG. 5B).

The aim of the present study was to develop immobilized sDBH and mDBH-based liquid chromatographic phases that could be attached on-line to HPLC analytical columns for screening of DBH inhibitors. The DBH-IAM and DBH-Glut-P stationary phases were prepared and packed into columns. The immobilized enzyme reactors (DBH-IAM-IMER and DBH-Glut-P-IMER) through the use of switching valve technology were separately linked to a phenylboronic acid column and coupled analytical columns. The resulting IMER) retained their catalytic activities displaying distinct sensitivity to pH, temperature and inhibitors.

Experimental

Materials

Dopamine-beta-hyrdoxylase (from bovine adrenals), catalase (from bovine liver), DL-octopamine hydrochloride, tyramine hydrochloride, (±)-norepinephrine bitartrate salt, (−)-norepinephrine bitartrate salt, dopamine hydrochloride, fumaric acid, fusaric acid, captopril, ascorbic acid, and other chemicals unless otherwise stated were obtained from Sigma Chemical Company (St. Louis, Mo., USA). Glacial acetic acid, HPLC grade, was manufactured by J. T. Baker (Phillipsburg, N.J., USA) and purchased through Moquin Scientific (Montreal, QC, Canada). The IAM.PC (12 μm, 300 Å) non-endcapped chromatographic support was obtained from Regis Chemical Co. (Morton Grove, Ill., USA). The IAM.PC bonded phase, according to the manufacturer, contains a near monolayer of C14 saturated phosphatidylcholine, covalently linked to silica through an amide link. Glutaraldehyde-P affinity packing (40 μm, 300 Å) was obtained from J. T. Baker Inc.

Instrumentation and Operating Conditions

Three modular HPLC systems were setup in order to carry out on-line hydroxylation of tyramine by the DBH-IMERs. System 1 consisted of a Thermo Separation Products P1000 pump (ThermoQuest, San Jose, Calif., USA), a Rheodyne 7125 injector with a 100 pd sample loop (Rheodyne, Cotati, Calif., USA), and the DBH-IMER of interest. System 2 consisted of a Thermo Separation Products P2000 binary pump and a phenylboronic acid column(PBA). System 3 consisted of a Thermo Separation Products P1000 pump, a 5□m octadecyl (ODS) stationary phase packed in a 250×4.6 mm column (Regis Chemical Co. Morton Grove, Ill.) connected in series, a SpectraSystem FL2000 fluorescence detector, and a Thermo Separation Products Chromjet integrator interfaced with a computer equipped with WOW software for data collection. The eluent from system 1 was directed onto system 2 then onto system 3 through Rheodyne 7000 switching valves (SV).

System 3 was used independently of systems 1 and 2 by replacing the latter systems with a Rheodyne 7125 injector (1) in order to analyze the results obtained from incubations involving non-immobilized DBH and DBH immobilized onto the loose Glut-P stationary phase. For the temperature studies, the DBH-IMER temperature was controlled with a Fiatron System CH-50 Column Heater (Fiatron, Wis., USA).

Chromatographic Conditions

The mobile phase on system 1 consisted of sodium acetate buffer (10 mM at the appropriate pH for each DBH-IMER) with a flow rate of 0.3 ml/min. System 3 contained two mobile phases A and B. Mobile phase A consisted of sodium phosphate buffer (25 mM, pH 8.4 and mobile phase B consisted of sodium phosphate buffer (25 mM, pH 4). A mobile phase consisting of potassium phosphate buffer (25 mM) adjusted to pH 2.0 with trifluoroacetic acid was utilized for system 3 to achieve the desired chromatographic separation of the products from the substrates. The solutes were quantitated using fluorescence detection with excitation at λ=266 nm and emission at at λ=380 nm. A flow rate of 0.7 ml/min and ambient temperature were used for system 2 throughout the study.

Enzyme Immobilization on Loose Packing Material

Immobilization of DBH onto LAM

DBH was immobilized onto IAM stationary phase utilizing a previously described method (Markoglou et al., 2001). IAM stationary phase (200-250 mg) was washed five times with sodium acetate buffer (0.1M, pH 5.5). The washing was carried out by adding 2 ml of buffer to the packing material, the suspension was centrifuged at 3000×g for 5 min and the supernatant decanted. The enzyme solution (1.65 mg in 2 ml sodium acetate buffer, 0.1M, pH 5.5) was added to the stationary phase, the mixture was placed in a rotator/stirrer for 12 h at ambient temperature. At the end of 12 h, the suspension was centrifuged for 5 min, the supernatant was collected and the stationary phase was washed an additional five times with buffer. The amount of enzyme immobilized was determined by measuring the amount of residual enzyme present in the supernatant using the Bio-Rad Protein Assay (Bio-Rad Laboratories Ltd, Mississauga, Ontario, Canada). The difference in the absorbance reading before immobilization and the combined absorbances of the washings after immobilization determined the amount of enzyme bound on the IAM stationary phase.

Immobilization of DBH onto the Glut-P Interphase

The immobilization of DBH onto the Glut-P interphase involved a similar approach to that utilized for immobilization onto IAM. The washing of the stationary phase involved the addition of 2 ml of sodium acetate buffer to the material (0.1M, pH 6). The enzyme solution (1.65 mg in 2 ml sodium acetate buffer, 0.1M, pH 6.0) was added to the stationary phase, the mixture was placed in a rotator/stirrer for 12 h at ambient temperature. The amount of enzyme immobilized was measured utilizing a similar approach described for immobilization onto LAM material.

Preparation of DBH Immobilized Enzyme Reactors

DBH immobilized on the IAM or Glut-P stationary phase was packed into a 1 cm×10 mm guard (Regis Technologies). The guard was placed in a holder and the resulting column connected to a chromatographic system. THE DBH-IAM-IMER and the DBH-Glut-P-IMER was washed with sodium acetate buffer [0.1M, pH 5.5] and sodium acetate buffer [0.1M, pH 6.0], respectively. The eluent from both IMERs was collected in order to determine if any of the DBH was being washed off the columns. The Biorad assay was utilized to measure the amount of non-immobilized enzyme. When the columns were not in use they were washed with sodium acetate buffer at the respective pHs and stored at 4° C.

Procedure for On-Line Injection

DBH-IAM-IMER and DBH-Glut-P-IMER

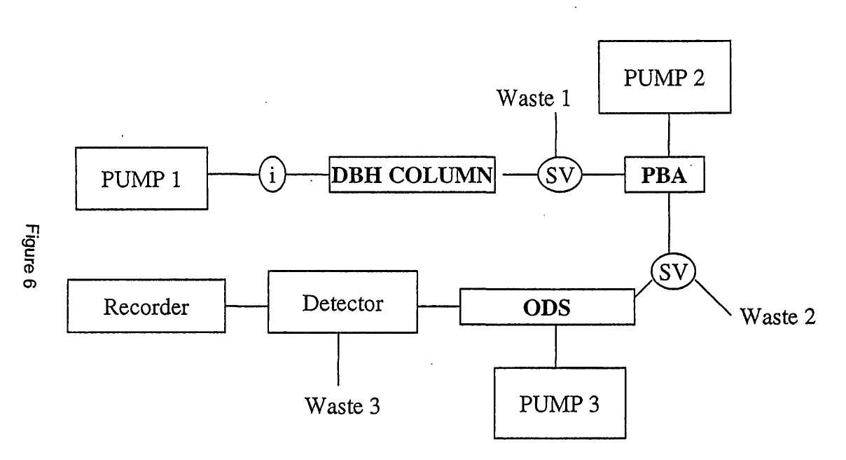

A schematic diagram of the coupled HPLC system is presented in FIG. 6. Pumps 2 and 3 on system 2 and 3 were stopped. 100 μl of a substrate/cofactor mixture is loaded into the injector (i) and the valve is switched to the inject position at the same time the witching valve (SV) is switched such that substrate/product are eluted from the DBH-IMERs and concentrated onto the PBA column of system 2.The second pump was started and mobile phase A was pumped through the PBA column at a flow rate of 0.1 ml/min for 30 sec in order to elute, ascorbic acid and any other by-products produced from the reaction. The pump was then switched to mobile phase B with a flow rate of 0.1 ml/min for 2 min and simultaneously SV1 was switched such that unreacted substrate and product formed were concentrated on the analytical columns of System 3. When the specific contact time elapses the SV2 is switched back to the original position and the pump 3 is started.

Effect of Flow Rate and Contact Time on On-Line System

The effect of flow rate through the DBH-Glut-P-IMER and DBH-IAM-IMER was investigates at flow rates ranging from 0.1 to 1.0 ml/min at 0.1 ml increments yielding a fixed elution volume of 3 ml at the respective flow rates.

The effect of contact time through the DBH-IMERs was also investigated at a fixed flow rate of 0.2 ml/min. Contact times from 5 to 30 min were investigated at 5 min increments.

The effect of flow rate and contact time was also examined on the PBA column to determine conditions that would yield maximal recovery of unreacted substrate and product formed. Flow rates ranging from 0.1 to 0.4 ml/min and contact times of 30 sec to 3 min were examined.

Effect of pH and Temperature on the DBH-IMERs

The activities of the DBH-IMERs were measured at a series of pHs (with 0.1M buffers) to determine the optimum pH. The temperature of the IMERs was kept at 37° C.

The effect of temperature was also examined for the individual DBH-IMERs using temperatures ranging from 25° C. (RT) to 60° C.

Enzyme Activity and Inhibition Studies on DBH-Glut-P-IMER

The enzymatic activity on the DBH-Glut-P was determined by quantification of the amount of product formed with a given substrate. The temperature of the IMER unless otherwise stated was kept at 37° C. with a column heater. Stock solutions of tyramine were prepared in water. The substrate concentrations examined ranged from 0.1-10 mM and that of the cofactor, ascorbic acid, ranged from 1-10 mM. Enzymatic activity was examined carrying out injections of a series of substrate/cofactor mixtures. The mixtures were injected onto the DBH-IMER at a flow rate of 0.3 ml/min for a contact time of 10 min. The kinetic parameters were determined using standard Michaelis-Menten approach. Lineweaver-Burke plots were used to calculate the Michaelis constant (Km). The rates of reaction (Vmax) were calculated using □mol/mg/min. Results are expressed as mean±standard error of the mean (SEM).

The effect of known inhibitors, fusaric acid and captopril on the enzymatic activity of the DBH-Glut-P IMER was also examined. The inhibition of the IMER was carried out using injections of a series of substrate/cofactor/inhibitor mixtures.

Results and Discussion

DBH was previously reported to be immobilized covalently onto Glut-P silica based chromatographic phase and immobilized by hydrophobic entrapment onto IAM stationary phase. In this study, 0.76±0.21 mg of DBH was immobilized onto 320±3.3 mg of Glut-P and packed into a column to form the DBH-Glut-P-IMER. The DBH-LAM-IMER was formed in a similar manner with 0.89±0.40 mg of DBH immobilized onto 305±4.7 mg of LAM. These DBH interphased have been formatted into a flow systems.

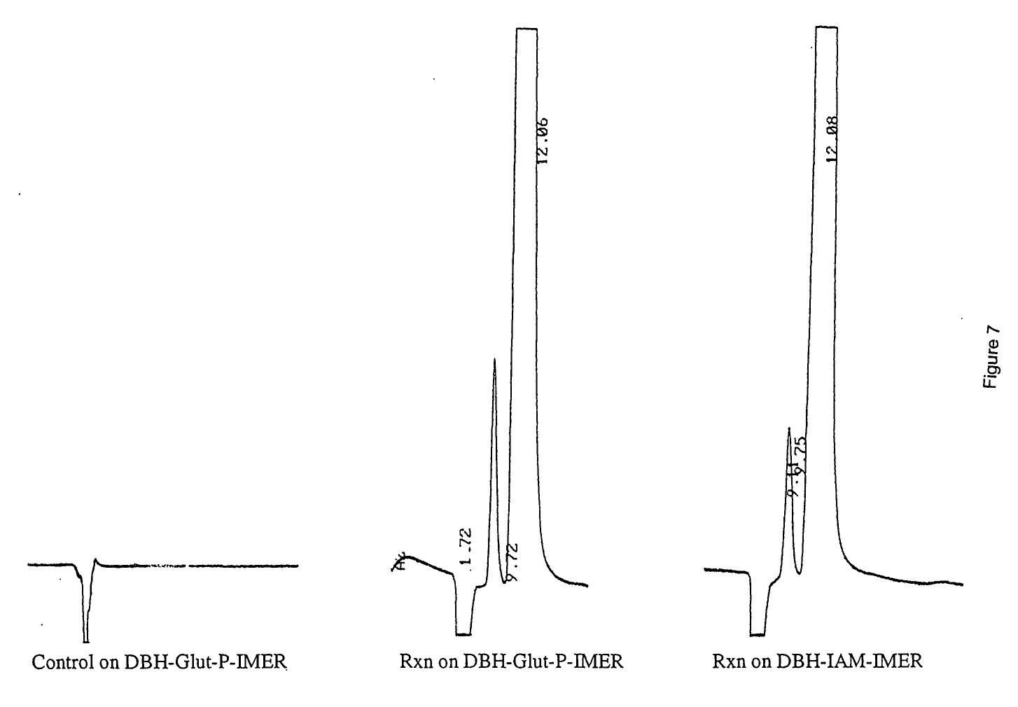

Immobilized DBH in the flow systems was shown to be active. Chromatographic studies with the two IMERs are depicted in FIG. 7. A mixture of tyramine and ascorbic acid was injected onto the DBH-IMER and the eluent form the IMER s were concentrated onto system 2 containing a PBA column for on-line extraction of ascorbic acid and any by-products produced during catalysis. Unreacted substrate and product are then concentrated onto coupled analytical columns for separation and analysis. FIGS. 7B and C display typical chromatographic profiles achieved on the DBH-Glut-P-IMER and the DBH-IAM-IMER respectively. A postitive control was carried out by injecting tyramine onto the system without the presence of the cofactor, ascorbic acid. No product formation was observed under these conditions as seen in FIG. 7A.

Optimal conditions of flow rate and contact time had to be determined for each subset (i.e. systems 1 through 3) of the on-line system in order to achieve the maximal productivity. A flow rate of 0.3 ml/min through both IMERs with a contact time of 10 min resulted in the maximal recovery of the product formed. Extraction of the cofactor and other by-products was achieved on-line through the use of the PBA column. At a flow rate of 0.1 ml/min and a contact time of 30 sec over 95% of the unreacted substrate and product were extracted on-line.

The kinetic parameters, Km and Vmax was determined for the DBH-Glut-P IMER. For the substrate, tyramine, the observed Vmax was reduced by approximately half and the Km remained similar to the non-immobilized enzyme, Table 3. Comparisons of the Km and Vmax values obtained with the DBH-Glut-P-IMER and the DBH-Glut-P-SP shows a reduction of the affinity and activity of the DBH-Glut-P IMER. The rate at which the substrate reaches the active site of the enzyme is affected by the experimental format. The enzyme kinetics alter from a non-flowing system to a flowing system.

TABLE 3
Kinetic Parameters for Non-Immobilized,
Immobilized DBH (DBH-SP) and DBH Immobilized
Enzyme Reactor (DBH-Glut-P IMER)

| | Km (mM) | Vmax (μmol/mg/min) |
|---|---|---|
| Non-immobilized DBH | | |
| Tyramine | 2.85 | 0.208 |
| Ascorbic acid | 0.62 | 0.185 |
| DBH-Glut-P-SP | | |
| Tyramine | 1.04 | 0.112⁻ |
| Ascorbic acid | 1.10 | 0.047 |
| DBH-IMER | | |
| Tyramine | 2.76 | 0.079 |
| Ascorbic acid | 0.54 | 0.084 |

The ability to examine DBH activity on an on-line chromatographic system allows for the examination of possible inhibitors of the enzyme. The effect of fusaric acid and captopril, known inhibitors of DBH, was examined on the DBH-Glut-P IMER. Fusaric acid and captopril were shown to inhibit the IMER at concentrations as low as 3 nM and 50 μM, respectively. The IMER can therefore allow for the screening and characterization of potent inhibitors.

TABLE 4
The Effect of Known Inhibitors on the Activity
of Non-Immobilized DBH and the DBH Immobilized Enzyme
Reactor (DBH-IMER)

| | DBH-Glut-P IMER | | DBH (Non-immobilized) | |
|---|---|---|---|---|
| | IC₅₀ | | IC₅₀ | |
| Captopril | 120 | μM | 150 | μM |
| Fusaric Acid | 5.6 | nM | 7.5 | nM |

Previous findings have demonstrated the DBH-LAM and DBH-Glut-P interphases are representative of the membrane-bound and soluble enzyme (Markoglou and Wainer, 2001). In this study, two individual IMERs, DBH-Glut-P-IMER and DBH-IAM-IMER were prepared and formatted onto an on-line system for the synthesis of octopamine from tyramine. Both IMERs can be used on the system for the generation, separation and identification of inhibitors and substrates. The individual IMERs will prove useful for the screening of substances for their pharmacological properties for membrane bound and soluble forms of DBH.

### EXAMPLE 3

### SYNTHESIS AND CHARACTERIZATION OF AN IMMOBILIZED PHENYLETHANOLAMINE N-METHYLTRANSFERASE LIQUID CHROMATOGRAPHIC STATIONARY PHRASE

Phenylethanolamine N-methyltransferase (PNMT) is the enzyme responsible for the N-methylation of norepinephrine to epinephrine. Enzymatic activity requires the presence of a hydroxyl group beta to the amino moiety and S-adenosyl-L-methionine as a methyl donor (Boulton et al., 1996). PNMT displays poor substrate specificity, transferring the methyl group to the nitrogen atom of a variety of β-hydroxylated amines (Grunewald et al., 1992).

Numerous methods have been developed to assay PNMT activity. Radiochemical methods and chromatographic methods coupled to electrochemical detection are among the most commonly used analytical methods (Molinoff et al., 1969; Ray et al., 1979; Saavedra et al., 1974; Trocewisz et al., 1982; Vogel et al., 1976). However, these methods either require radioactive substrates and/or large amounts of purified enzyme. In addition, these methods use solubilized enzymes and tend to be complicated, costly and time-consuming.

One way to circumvent these limitations is to provide enzyme in an immobilized form. An example of this approach is the work by Wainer et al. who have developed immobilized enzyme reactors based upon α-chymotrypsin (Chui et al., 1992; Jadaud et al., 1989; Wainer et al., 1998), trypsin (Thelohan et al., 1989), lipase (Johnson et al., 1997; Sotolongo et al., 1999; Zhang et al., 1993), alcohol dehydrogenase (Alebic-Kolbah et al., 1993; Alebic-Kolbah et al., 1993) β-glucoronidase (Pastemyk Di Marco et al., 1998; Pastemyk et al., 1998) and cytochrome P450s (Alebic-Kolbah et al., 1993; Alebic-Kolbah et al., 1993). These immobilized enzyme reactors have proven useful for the on-line or batch-wise generation, separation and identification of metabolites as well as for the identification of inhibitors.

In these studies, α-chymotrypsin was covalently immobilized onto a silica based liquid chromatographic stationary phase, glutaraldehyde-P (Glut-P) The immobilization was accomplished through the formation of a Schiff-base between an amine group on the α-chymotrypsin molecule and a glutaraldehyde moiety covalently linked to the stationary phase. The resulting liquid chromatographic stationary phase was shown to be enzymatically active and capable of the on-line liquid chromatographic stereochemical resolution of substrate analog amino acids and amino acid deriavatives.

For the first time we show and use of the glutaraldehyde-P liquid chromatographic stationary phase for the covalent immobilization of PNMT. The resulting PNMT-Glut-P stationary phase (PNMT-SP) is stable and capable of the transmethylation of normetanephrine. Standard Michaelis-Menten kinetic studies were carried out for both free and immobilized PNMT. Known substrates and inhibitors for PNMT were examined, and the results demonstrate that the PNMT-SP can be utilized for both qualitative and quantitative determinations of enzymatic activity in batchwise (i.e. non-flow) and flow formats. The PNMT-SP can be utilized for rapid screening of potential PNMT substrates and inhibitors.

Materials and Methods

Materials

Phenylethanolamine-N-methyltransferase, S-adenosyl-L-methionine, DL-normetanephrine hydrochloride, DL-metanephrine, benzylamine hydrochloride, N-ethylmaleimide, p-chloromercuriphenylsulfonic acid monosodium salt and other chemicals unless otherwise stated were obtained from Sigma Chemical Co. (St. Louis, Mo., USA). Glutaraldehyde-P 40 μM affinity packing, 300 Å was obtained from J. T. Baker Inc. (Phillipsburg, N.J., USA).

Apparatus

The chromatographic experiments were carried out using a Thermo Separation Products P1000 pump, a Thermo Separation Products AS3000 autosampler equipped with a 100 μl loop, a SpectraSystem FL2000 fluorescence detector and data collection was carried out using a Thermo Separation Products Chromjet integrator interfaced with a computer equipped with WOW software for data collection (ThermoQuest, San Jose, Calif., USA). The chromatographic separations were performed using a 5 μm phenyl stationary phase packed in 150×4.6 mm column (Regis Chemical Co. Morton Grove, Ill.) and a 5 μm C18 stationary phase packed in 250×4.6 mm column (Regis Chemical Co.) connected in series.

Chromotographic Procedures

A mobile phase consisting of potassium phosphate buffer (50 mM) adjusted to pH 2.0 with trifluoroacetic acid was utilized to achieve the desired chromatographic separation of the products from the substrates. The solutes were quantitated using fluorescence detection with excitation at λ=266 nm and emission at λ=380 nm. A flow rate of 0.7 ml/min and ambient temperature were used throughout the study.

Assay for Phenylethanolamine N-methyltransferase Activity

The activity of phenylethanolamine N-methyltransferase was assayed as follows: [final concentration] To 500 μL of enzyme solution [163 μg] was added 50 μL S-adenosyl-L-methionine [20 μM] and the solution was vortexed for 1 min. The reaction was started by addition of the 50 μL substrate [1 mM]. The reaction medium was incubated for 10 min at 37° C. in a shaking bath. The resulting solution was centrifuged at 3000×g and the supernatant were directly injected onto the HPLC under the above mentioned conditions.

Covalent Immobilization of PNMT

Immobilization onto the Glut-P Liquid Chromatographic stationary phase was accomplished in the following manner: (1) the stationary phase (10-100 mg) was washed three times with 0.1 M sodium phosphate buffer adjusted to pH 8.30 with 5M NaOH. The washing was carried out by adding 1 ml of buffer to the stationary phase, the suspension was mixed for 1 min, centrifuged at 3000×g for 10 min, and the supernatant decanted. (2) The enzyme solution [98 μg in 0.6 ml sodium phosphate buffer (0.1M, pH 8.30)] was added to the packing material, the mixture was mixed gently for 5 min and then placed in a rotator/stirrer bath for 18 h at ambient temperature. (3) At the end of 18 h, the suspension was centrifuged, the supernatant decanted and the packing material was washed three additional times with buffer. (4) The amount of enzyme immobilized on the packing material was determined by measuring the amount of residual enzyme in the supernatant using the BioRad Protein Assay. The difference in the absorbance reading before immobilization and the combined absorbances of the washings after immobilization determined the amount of enzyme bound on the packing material.

Regeneration of Enzyme Activity

A simple washing procedure was utilized to regenerate the activity of the immobilized enzyme. To the PNMT-Glut-P material was added 1 ml of sodium phosphate buffer [0.1M, pH 8.30]. The mixture was mixed for 1 min, centrifuged at 3000×g for 10 min, and the supernatant was discarded. The PNMT-Glut-P material was stored in sodium phosphate buffer [0.1M, pH 8.30] at 4° C. and remained active. When the enzyme was stored at room temperature for an 18-day period almost 75% of the enzyme activity was lost. However, storage of the material at 4° C. retained over 85% enzymatic activity for over a three-month period.

Results

Chromatographic Results

Using the chromatographic conditions described here, normetanephrine and metanephrine were resolved from each other with relative retentions, k′, of 7.63 and 13.04, respectively. Standard curves for metanephrine ranged from 1.25×10⁻⁵ mM to 1 mM and for normetanephrine ranging from 6.25×10⁻³ mM to 10 mM. Standard curves for the substrate and product were linear over the range investigated. The results demonstrate that the product enzymatically formed by immobilized PNMT can be isolated by HPLC and determined by fluorescence detection. A boiled enzyme control preparation and the reaction mixture alone contained no interfering peaks.

Optimization of PNMT Immobilization

The amount of PNMT immobilized onto the Glut-P stationary phase was examined using PNMT (98 μg in 0.6 ml of sodium phosphate buffer [0.1M, pH 8.30] and 10-100 mg of Glut-P. The amount of immobilized enzyme and the rate of the reaction were investigated for the different amounts of Glut-P packing material. When greater than 50 mg of Glut-P were used to immobilize 163 μg of PNMT, over 80% of the enzyme was immobilized i.e. 135±0.16 μg of PNMT was immobilized on 50 mg of the Glut-P material. However, there was a decrease in the enzymatic activity when greater than 50 mg of the Glut-P material was used. The optimal conditions were found to be 163 μg of DBH being immobilized onto 50 mg of Glut-P for the remainder of the experiments.

Effect of Incubation and Enzyme Concentration on PNMT Activity

The optimal assay conditions for both forms of the enzyme were determined by varying independently the length of incubation and amount of enzyme. The optimal conditions were determined to be a 10-min incubation utilizing 120-250 μg of phenylethanolamine N-methyltransferase. The amount of phenylethanolamine N-methyltransferase that was prepared for each experiment was determined utilizing the BioRad assay each time. The activity of free and immobilized PNMT as measured with the substrate normetanephrine, is linear up to 250 μg of enzyme under the assay conditions.

Effect of pH on PNMT Activity

The activity of the free and of the immobilized phenylethanolamine N-methyltransferase was measured at a series of pHs (with 0.1M ionic strength buffers) to determine the optimum pH. A pH optimum of 8.30 was found for both the immobilized and non-immobilized enzyme.

Effect of Substrate and Cofactor Concentration on PNMT Activity

The effect of normetanephrine concentrations on the enzymatic activity of non-immobilized and immobilized PNMT was examined. The standard Michaelis-Menten approach was utilized to determine kinetic parameters for both forms of the enzyme. The results are shown in Table 5.

Table 1: Kinetic parameters for both the immobilized and non-immobilized PNMT.

TABLE 5
Effect of known inhibitors on
immobilized and non-immobilized PNMT.

| | PNMT | PNMT-SP |
|---|---|---|
| Normetanephrine | | |
| Kₘ (mM) | 0.109 | 0.152 |
| Vₘₐₓ (μmol/mg/min) | 1.136 | 0.823 |
| SAM | | |
| Kₘ (μM) | 14.17 | 10.56 |
| Vₘₐₓ (μmol/mg/min) | 1.249 | 0.654 |

Effect of Temperature on PNMT Activity

The effect of temperature was examined for both the free and immobilized enzyme. The non-immobilized enzyme was shown to have optimal activity at 60° C. after which the activity decreased with increasing temperature. The PNMT-Glut-P interphase however displayed optimal activity at 37° C. with little change at increased temperatures. The Arrhenius plots for both forms of PNMT were drawn. Data yielded straight lines yielding activation energies of 11.04 kJ/mol and 7.61 kJ/mol for the non-immobilized and immobilized PNMT, respectively.

Effect of Inhibitor Concentration on PNMT Activity

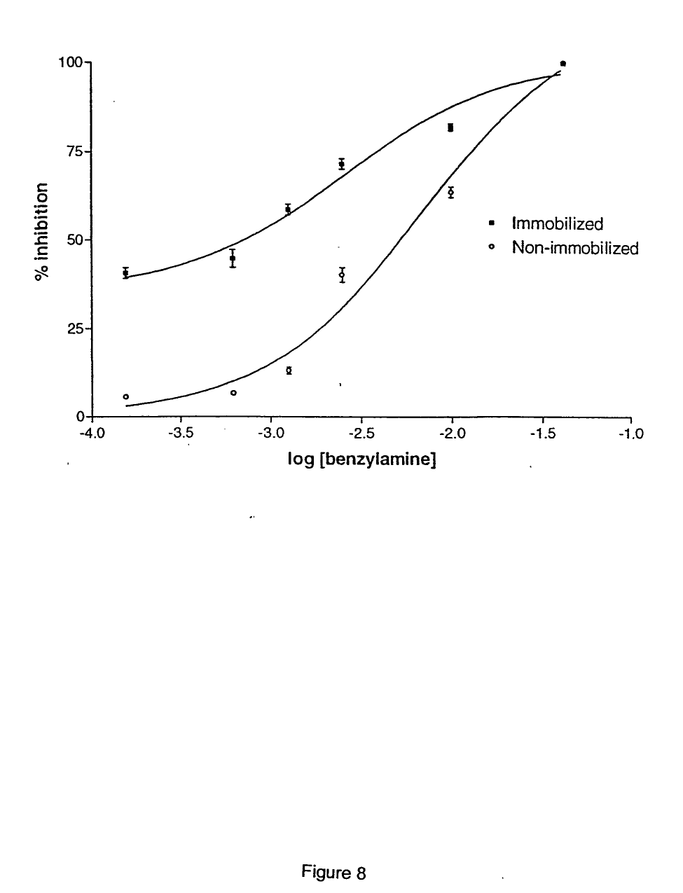

Benzylamine, p-chloromercuriphenylsulfonic acid, and N-ethylmaleimide are known inhibitors of phenylethanolamine N-methyltransferase (Grunewald et al., 1999). The effect of these compounds on the enzymatic activities of non-immobilized and PNMT-Glut-P was examined. The inhibition of PNMT activity by benzylamine was found to be three times higher with the immobilized PNMT compared to the non-immobilized PNMT (FIG. 8). Similarly, the inhibitory effects of N-ethylmaleimide and p-chloromercuriphenylsulfonic acid were found to be higher for the immobilized form of the enzyme (Table 6).

TABLE 6
Effect of Known Inhibitors on Immobilized
and Non-Immobilized PNMT

| | PNMT | PNMT-SP |
|---|---|---|
| Inhibitor | Ki | Ki |
| Benzylamine | 0.22 ± 0.11 mM | 0.69 ± 0.10 mM |
| N-ethylmaleimide | 1.04 ± 0.49 μM | 4.92 ± 0.16 μM |
| p-chloro- | 0.12 ± 0.14 mM | 0.52 ± 0.19 mM |
| mercuriphenylsulfonic acid | | |

Discussion

In this study PNMT has been immobilized onto a glutaraldehyde-P stationary phase and the enzyme remained active and retained its enzymatic characteristics. The kinetic parameters for the substrate and cofactor were determined for the immobilized and non-immobilized enzyme. The results obtained on the PNMT-SP are comparable to those obtained with the non-immobilized enzyme although the observed Vₘₐₓ and Kₘ values for the immobilized enzyme (Table 5) were lower relative to the non-immobilized enzyme. The relative differences in these enzymes may be due to the restrictions imposed by the immobilization. For example, the microenvironment of the immobilized enzyme can impede the rate at which the substrate and cofactor reach the active site. The conformational mobility of the enzyme may also be hindered by the covalent attachment to the chromatographic support.

The PNMT-Glut-P interphase displayed a similar pH optimum to that of the non-immobilized enzyme. Under the conditions utilized the optimal pH for activity was found to be 8.30 for both forms of the enzyme. As the non-immobilized enzyme is heated past 60° C. the enzyme activity is markedly decreased. A decrease in the activity is due to the thermal denaturation of the enzyme. However, for the immobilized enzyme optimal activity is visible at 37° C. with very little change in the rate at the higher temperatures. Arrhenius plots for both enzyme forms displayed continuous profiles. Activation energies of the free and immobilized PNMT were 11.04 kJ/mol and 7.61 kJ/mol, respectively.

Thermal movement of molecules at higher temperatures is limited due to the immobilization of the enzyme. As a result, thermal denaturation is not visible at the higher temperatures for the immobilized enzyme. Similarly, previous reports have demonstrated the protective effect of S-adenosyl-L-methionine on the heat inactivation of the enzyme. The microenvironment of the immobilized enzyme may amplify the protective effect of S-adenosyl-L-methionine.

The correct method of immobilization and choice of support is important when comparing free versus Immobilized enzyme. PNMT is localized in the soluble fraction of the adrenal medulla When PNMT is immobilized on the Glut-P interphase the enzyme is outside the stationary phase and not embedded within the interphase surroundings. As such this form of immobilization is comparable to the non-immobilized cytosolic enzyme.

Several phenylethylamines, benyzlamines and numerous conformational analogs of these compounds are used as in vitro and in vivo inhibitors of PNMT (Grunewald et al., 199). The inhibitory effect of three PNMT inhibitors was investigated for both the immobilized and non-immobilized enzyme. Benzylamine was capable of inhibiting both types of enzymes at concentrations as low as 1×10⁻⁴M. Sulfhydryl reagents such as N-ethylmaleimide and p-chloromercuriphenylsulfonic acid were shown to inhibit both types of enzyme. These compounds are well-known in vitro inhibitors of PNMT because the enzyme in known to possess an essential sulfhydryl group (Boulton et al., 1996).

The results herein demonstrate that the PNMT-SP can be utilized to screen for potent and selective inhibitors of the enzyme. In this study, PNMT-SP was used in a batchwise (non-flow) format. Based upon our results, the immobilized PNMT-SP has been formatted for a flow system. Thus, an in-line immobilized enzyme reactor based upon the Glut-P stationary phase has been developed and attached to an HPLC analytical column. This on-line system will prove to be a vital pharmacological tool for the development of potent PNMT inhibitors with minimal α₂-adrenoceptor binding affinity.

### EXAMPLE 4

### BIOSYNTHESIS USING AN ON-LINE IMMOBILIZED ENZYME REACTOR CONTAINING PHENYLETHANOLAMINE N-METHYLTRANSFERASE IN SINGLE ENZYME AND COUPLED ENZYME FORMATS

The PNMT-Glut-P stationary phase (PNMT-SP) was prepared as in EXAMPLE 3 and packed into a column. The resulting immobilized enzyme reactor (PNMT-IMER) was linked to coupled analytical HPLC columns through a switching valve and used for on-line N-methylation of known substrates of PNMT. The PNMT-IMER retained its catalytic activity and displayed sensitivity to pH, temperature and inhibitors. The results demonstrate that the PNMT-IMER can be utilized as a chromatographic probe of enzyme/substrate and enzyme/inhibitor interactions. The HPLC system allows for the generation, separation and identification of substances as well as the identification of inhibitors.

The utilization of the PNMT-IMER was expanded by coupling it to a DBH-IMER). The coupled system was shown to be capable of carrying on-line synthesis of epinephrine from dopamine in a continuous flow system. The immobilized enzyme reactors used independently or as a combination provides a unique opportunity to explore the interrelationships between these enzymes.

Experimental

Chemicals

Phenylethanolamine N-methyltransferase (from bovine adrenal medulla), s-adenosyl-L-methionine p-toluenesulfonate salt (SAM), DL-normetanephrine hydrochloride, DL-metanephrine hydrochloride, S-adenosyl-L-homocysteine (SAH), methyl-dopa, dopamine, norepinephrine, epinephrine and other chemicals unless otherwise stated were obtained from Sigma Chemical Co. (St. Louis, Mo., USA). Glutaraldehyde-P 40 μM affinity packing 300A was obtained from J. T. Baker Inc. (Phillipsburg, N.J., USA). A 1 cm phenylboronic acid cartridge from Varian Inc.(Palo Alto, Calif., USA) was used for on-line extraction for the coupled IMER system.

Instrumentation and Operating Conditions

PNMT-IMER System

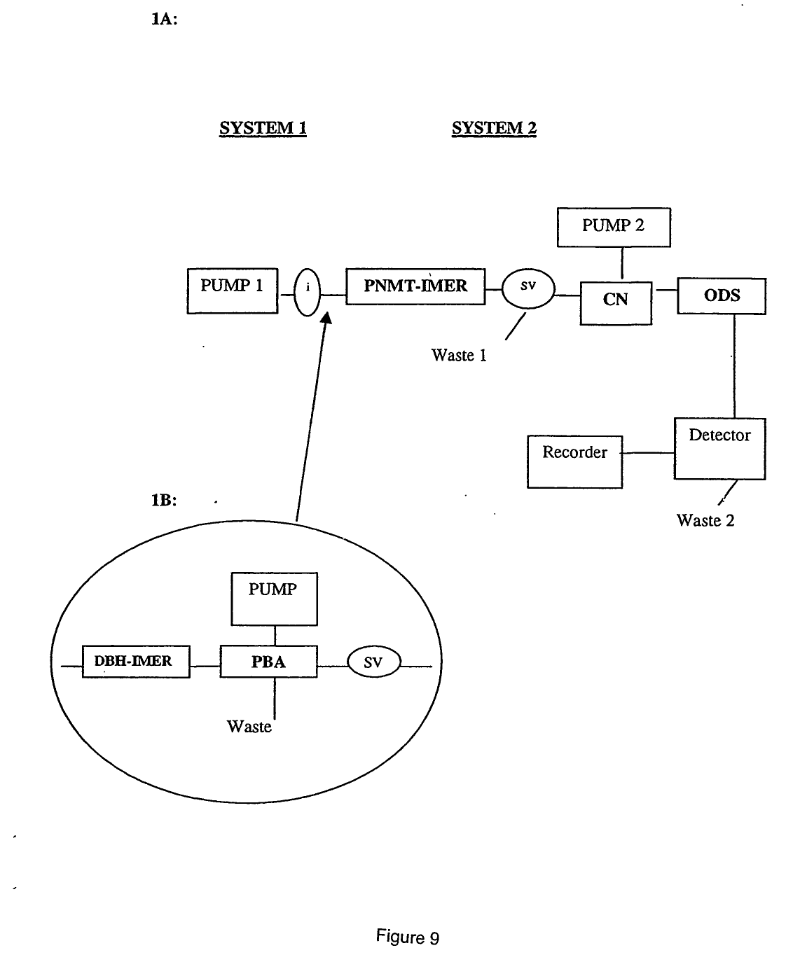

Two modular HPLC systems were setup in order to carry out the chromatographic experiments (FIG. 9). System 1 consisted of a Thermo Separation Products P1000 pump (ThermoQuest, San Jose, Calif., USA), a Rheodyne 7125 injcetor with a 100 μl sample loop (Rheodyne, Cotati; Calif., USA), and the PNMT-IMER System 2 consisted of a Thermo Separation Products P1000 pump, a 5 μm cyano (CN) stationary phase packed in 150×4.6 m column (Regis Chemical Co. Morton Grove, Ill.), as 5 μm octadecyl (ODS) stationary phase packed in a 250×4.6 mm column (Regis Chemical Co. Morton Grove, Ill.) connected in series, a SpectraSystem FL2000 fluorescence detector, and a Thermo Separation Products Chromjet integrator interfaced with a computer equipped with WOW software for data collection. The eluent from system 1 was directed onto system 2 through a Rheodyne 7000 switching valve (SV).

System 2 was used independently of system 1 by replacing the system with a Rheodyne 7125 injector (i) in order to analyze the results obtained from incubations involving non-immobilized PNMT and PNMT immobilized onto the loose Glut-P stationary phase. For the temperature studies, the PNMT-IMER temperature was controlled with a Fiatron System CH-50 Column Heater (Fiatron, Wis., USA).

Coupled IMER System

To the existing PNMT-IMER system was added the dopamine beta-hydroxylase immobilized enzyme reactor (DBH-IMER) coupled to a phenylboronic acid column (FIG. 9B). Previously reported instrumentation and operating conditions were used for the DBH-IMER

Chromatographic Conditions for PNMT-IMER System

The mobile phase on system 1 consisted of potassium phosphate buffer (0.1 M, pH 8.30) with a flow rate of 0.2 ml/min. A mobile phase consisting of potassium phosphate buffer (25 mM) adjusted to pH 2.0 with trifluoroacetic acid was utilized for system 2 to achieve the desired chromatographic separation of the products from the substrates. The solutes were quantitated using fluorescence detection with excitation at λ=266 nm and emission at λ=380 nm. A flow rate of 0.7 ml/min and ambient temperature were used for system 2 throughout the study.

Immobilization of PNMT on Loose Packing Material

PNMT was immobilized onto Glut-P stationary phase utilizing a previously reported method (Grunewald et al., 1999). Briefly, the following procedure was used: (1) the Glut-P stationary phase (300-350 mg) was washed five times with sodium phosphate buffer [0.1M, pH 8.3]. In this step, 2 ml of buffer was added to the stationary phase, the suspension was vortex-mixed for 15 min, centrifuged and the supernatant decanted. (2) The enzyme solution (1.96 mg in 2 ml sodium phosphate buffer [0.1M, pH 8.3D was added to the packing material, the mixture was mixed gently for 15 min and then placed in a rotator/stirrer for 24 h at ambient temperature. (3) At the end of 24 h, the suspension was centrifuged, the supernatant decanted and the packing material washed three additional times with buffer. (4) The amount of enzyme immobilized on the stationary phase was determined by measuring the amount of residual enzyme present in the supernatant using the Bio-Rad Protein Assay (Bio-Rad Laboratories Ltd, Mississauga, ON, Canada). The difference in the absorbance reading before immobilization and the combined absorbances of the washings after immobilization determined the amount of enzyme bound on the Glut-P stationary phase.

Preparation of PNMT Immobilized Enzyme Reactor

PNMT immobilized on the Glut-P stationary phase was packed into a lcm×10 mm guard (Regis Technologies). The guard was put into a holder and the column was placed onto the chromatographic system. The PNMT-IMER was washed with phosphate buffer [0.1M, pH 8.3]. The eluent was collected in order to determine if any of the enzyme was being washed off the column. The Biorad assay was utilized to measure the amount of non-immobilized enzyme. When the column was not in use it was washed with phosphate buffer [0.1M, pH 8.3] and stored at 4° C.

Procedure for On-Line Injection

PNMT-IMER

A schematic diagram of the coupled HPLC system is presented in FIG. 9. The pump on system 2 is stopped. 100 μl of a substrate/cofactor mixture is loaded into the injector (i) and the value is switched to the inject position at the same time the switching valve (SV) is switched such that the substrate/product are eluted from the PNMT-IMER and concentrated onto the analytical columns of System 2 for the specified contact time. When the specific contact time elapses the SV is switched back to the original position and the pump on system 2 is started. The unreacted substrate and product formed are separated on the coupled analytical columns.

Coupled IMERs

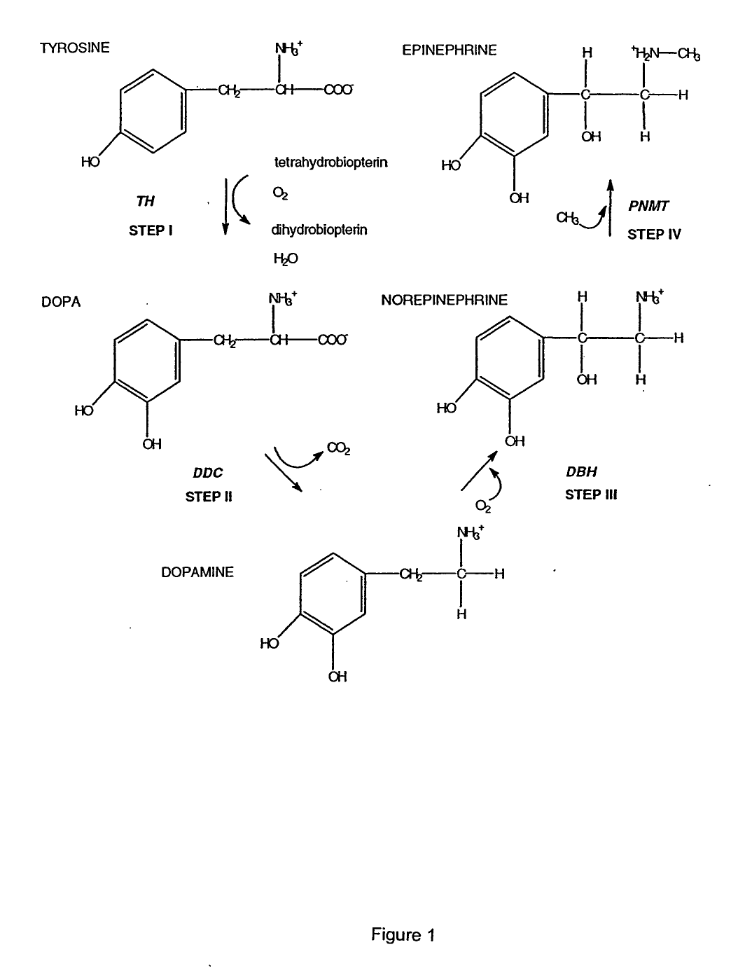

A representation of the coupled IMER system is illustrated by the incorporation of DBH-IMER (FIG. 9B) into the existing PNMT-IMER system (FIG. 1A). In order to carry out the on-line synthesis of epinephrine from dopamine the following procedure was used: The first pump connected to the DBH-IMER had a mobile phase of sodium acetate buffer [10 mM, pH 5.5] with a flow rate of 0.3 ml/min. All the other pumps in the system were stopped. 100 μl of a mixture of dopamine and ascorbic acid was loaded into the injector (i) and the valve position switched such that the substrate/product were eluted from the DBH-IMER onto the PBA column where they were trapped.

Following a contact time of 3 min, the second pump was started and mobile phase A [sodium phosphate buffer 25 mM, pH 8.4] was pumped through the PBA column at a flow rate of 0.1 ml/min for 30 sec in order to elute the cofactor, ascorbic acid, and any other by-products from the DBH catalyzed reaction. The pump was then switched to mobile phase B [sodium phosphate buffer 25 mM, pH 4] with a flow rate of 0.1 ml/min for 2 min and simultaneously SV1 was switched such that any unreacted substrate and product were eluted onto the PNMT-IMER. The PNMT-IMER system is treated as described above. The only consideration is the addition of SAM into the mobile phase of the pump connected to the PNMT-IMER. Unreacted dopamine and norepinephrine and epinephrine formed are concentrated and separated on the coupled analytical columns.

Effect of Flow Rate and Contact Time on the PNMT-IMER Activity

The effect of the flow rate through the PNMT-IMER was investigated at flow rates ranging from 0.1 to 0.4 ml/min at 0.1 ml/min increments. The contact time was 20 min yielding elution volumes of 2, 4, 6 and 8 ml at the respective flow rates.

The effect of contact time through the PNMT-IMER was also investigated at a fixed flow rate of 0.2 ml/min. Contact times from 5 to 30 min were investigated at 5 min increments. The recoveries of the substrate and product were determined.

Effect of pH and Temperature on the PNMT-IMER Activity

The activity of the PNMT-IMER was measured at a series of pHs (with 0.1M buffers) to determine the optimum pH. The temperature of the PNMT-IMER was kept at 37° C.

The effect of temperature was also examined for the PNMT-IMER using temperatures ranging from 25° C. (RT) to 60° C. The effect of temperature on PNMT-SP and the PNMT-IMER was compared using Student's t-test for unpaired data. All p values <0.05 were considered statistically significant.

Enzyme Activity and Inhibition Studies on PNMT-IMER

The enzymatic activity on the PNMT-IMER was determined by quantification of the amount of product formed with a given substrate. The temperature of the PNMT-IMER unless otherwise stated was kept at 37° C. with a column heater. Stock solutions of normetanephrine were prepared in water. The substrate concentrations examined ranged from 0.15-10 mM and that of the cofactor, S-adenosyl-L-methionine, ranged from 5-100 μM.

Enzymatic activity was examined carrying out injections of a series of substrate/cofactor mixtures. The mixtures were injected onto the IMER at a flow rate of 0.2 ml/min for a contact time of 20 min. The kinetic parameters were determined by drawing Lineweaver-Burke plots utilizing Microsoft Excel. Results are expressed as mean±standard error of the mean (SEM).

The effect of known inhibitors, S-adenosylhomocysteine and methyldopa on the enzymatic activity of the PNMT-IMER was also examined. The inhibition of the PNMT-IMER was carried out using injections of a series of substrate/cofactor/inhibitor mixtures.

Results and Discussion

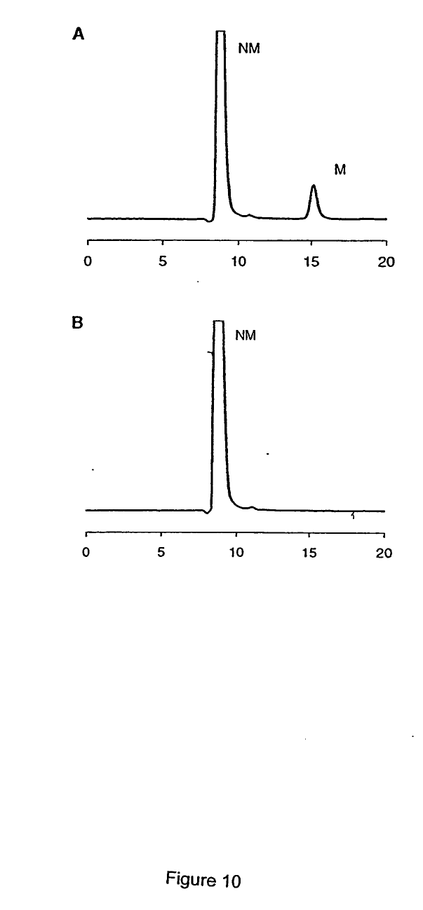

Chromatographic studies with the PNMT-IMER and the coupled-system depicted in FIG. 1, demonstrated that the immobilized PNMT is active in the flow system. When a mixture containing normetanephrine (NM) and S-adenosyl-L-methionine (SAM) was injected onto the PNMT-IMER and the eluent from the PNMT-IMER analyzed on system 2, M appeared in the chromatogram, FIG. 10A. As a positive control, NM was injected onto the coupled system without the cofactor, SAM. Under these conditions, no product formation was observed in the resulting chromatogram, FIG. 10B. The negative control consisted of a column packed with immobilized PNMT that had been heat inactivated before immobilization. Injections of NM/SAM mixtures onto the system containing the inactive PNMT-IMER did not result in the production of metanephrine (M). Thus, the production of M in this system was due to the activity of the PNMT-IMER.

The production of the PNMT-IMER depends upon the time that the substrate/cofactor mixture is in contact with the immobilized enzyme. Therefore, the flow rate through the PNMT-IMER is a key experimental variable. In order to optimize this factor, flow rates ranging from 0.1 to 0.4 ml/min at 0.05 ml/min increments were investigated using a fixed contact time of 20 min. A flow rate of 0.2 ml/min allowed for maximal recovery of product formed as well as any unreacted substrate.

The affinity (expressed as the Michaelis-Menten constant, Kₘ) and the enzymatic activity (expressed as maximum velocity, Vₘₐₓ) of the immobilized PNMT in the IMER format was determined. For the substrate, NM, the observed Kₘ value was increased and the Vₘₐₓ reduced, both by a factor of approximately 4, relative to the non-immobilized enzyme, Table 7. An increase in Kₘ indicates a reduced affinity while a decrease in Vₘₐₓ indicates a reduced activity. Thus, the immobilization of PNMT negatively affected the enzyme's activity. However, the magnitudes of the observed effects were not solely due to the immobilization of the enzyme. Comparisons of the Kₘ and Vₘₐₓ values obtained with the PNMT-IMER and the PNMT-SP also show a reduction of the affinity and activity of the PNMT-IMER relative to the PNMT-SP. In this case, the values differed by a factor of approximately three, Table 7.

TABLE 7
Kinetic parameters for the immobilized, non-
immobilized PNMT-PNMT-IMER (n = 2).

| | PNMT | PNMT-SP* | PNMT-IMER |
|---|---|---|---|
| Normetanephrine | | | |
| Kₘ (mM) | 0.109 | 0.152 | 0.384 |
| Vₘₐₓ (μmol/mg/min) | 1.136 | 0.823 | 0.292 |
| SAM | | | |
| Kₘ (μM) | 14.17 | 10.56 | 7.31 |
| Vₘₐₓ (μmol/mg/min) | 1.249 | 0.254 | 0.424 |

*Data obtained from reference 14.

The immobilization of an enzyme places the protein in a new microenvironment that can impede the rate at which the substrate reaches the active site of the enzyme. This is demonstrated by changes in the Kₘ and Vₘₐₓ values between the non-immobilized PNMT and the PNMT-SP, Table 7.

However, these values differ by less than 50%. Therefore, the magnitude of changes seen with the PNMT-IMER must be due to the experimental format i.e. the change from a non-flowing system (non-immobilized PNMT and PNMT-SP) to a flowing system (PNMT-IMER). In this case, the key factors may be the kinetics of the distribution of the substrate from the mobile phase to the stationary phase and the shearing forces produced by the moving phase.

The effect of temperature on the PNMT-IMER was also examined. The PNMT-IMER was shown to display maximum product formation at 37° C. with limited changes in production at higher temperatures. Both the PNMT-IMER and PNMT-SP displayed no significant difference in the amount of product formed at temperatures exceeding 37° C. The non-immobilized enzyme however shows a considerable decrease in production of M at temperatures exceeding 60° C. [14]. The increase in stability of the immobilized enzymes is due to the environment that the enzymes are subjected to upon immobilization. Upon immobilization the enzyme is restricted in movement, which account for the lack of thermal denaturation at the higher temperatures.

PNMT is known to be inhibited by its own substrates and products at certain concentrations. The inhibitory effect of two PNMT inhibitors, S-adenosyl-L-homocysteine (SAH) and methyldopa was investigated for both PNMT-IMER and non-immobilized enzyme. Fifty percent inhibition was achieved at similar concentrations for both enzyme forms see Table 8. The PNMT-IMER was shown to be inhibited by SAH at concentrations as low as 5 μM and methyldopa concentrations of 1 μM. The PNMT-IMER can therefore be used to designate the relative affinities of potential PNMT inhibitors.

TABLE 8
The effect of known inhibitors on the activity
of non-mmobilized PNMT (PNMT) and PNMT immobilized
enzyme reactor (PNMT-IMER) (n = 2).
IC 50

| Inhibitor | PNMT | PNMT-IMER |
|---|---|---|
| Methyl-dopa | 10.4 μM | 7.6 ± 0.2 μM |
| SAH | 40.1 μM | 50.5 ± 1.5 μM |

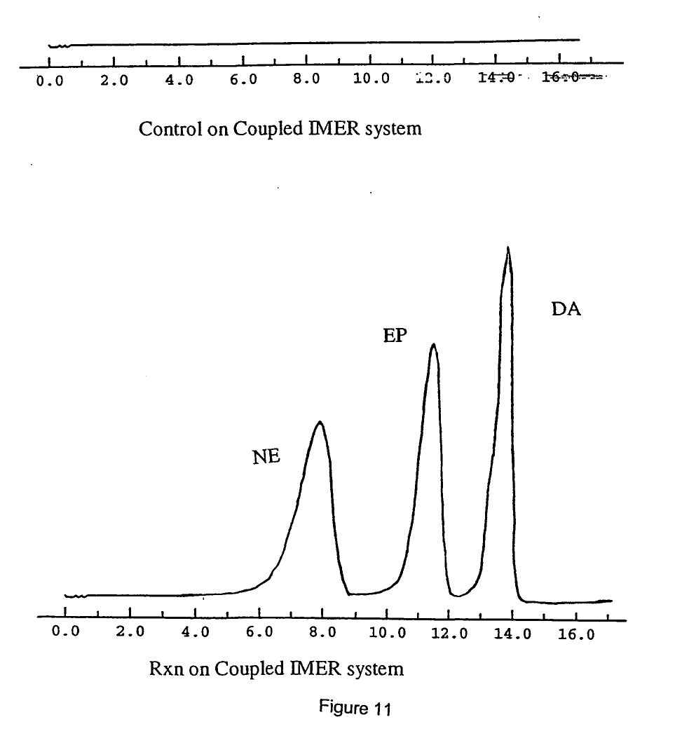

In this study, two individual IMERs based upon DBH and PNMT were coupled using switching valve technology and shown to carry out the on-line synthesis of epinephrine from dopamine (FIG. 11). Dopamine was injected onto the DBH-IMER and the reactants and products were eluted onto a phenylboronic acid column for on-line extraction. The substrates and products were transported via a switching valve to the PNMT-IMER. Norepinephrine was then converted into epinephrine by the PNMT-IMER and directed onto the analytical columns for analysis. The system allows for the analysis of the IMERs individually or as a combination. The construction of a coupled system of this nature provides a number of approaches to basic research into synthetic and metabolic pathways as well as a rapid method for the discovery of new pharmaceutical substances.

### EXAMPLE 5

### IMMOBILIZATION OF PNMT ONTO OPEN-TUBULAR COLUMNS

Numerous methods for immobilizing enzymes have been reported. The ideal method of choice is contingent upon numerous variables. The immobilized enzyme allows for its reuse and investigation into substrate/inhibitor interactions. The development of on-line systems that can carryout quantitative and qualitative determinations are important for the rapid discovery of drug candidates for various diseases. A technological revolution has occurred in recent years resulting in greater productivity, reduction in time and cost and the development of novel drugs. One key advance in high throughput screening has been miniaturization and parallel processing. In relation to the supports described herein, they can also be sealed down to create microscale analyzers. Microfabrication and micromachinery allows for the acceleration of large scale screening of potential inhibitors and substrates of the enzymes. Miniaturization of these processes could be useful for screening thousands of compounds that are generated by combinatorial chemistry techniques.

Based upon the results obtained from previous work (Markoglou and Wainer, 2001), PNMT has been immobilized onto an open tubular column utilizing a modified procedure described by Yang et al. (Yang et al., 1998). The open tubular column containing immobilized PNMT was coupled to a mass spectrometer for analysis. Results confirm that by immobilizing PNMT onto the capillary the enzyme remains active and retains its enzymatic characteristics.

Materials

Phenylethanolamine-N-methyltransferase, DL-metanephrine, S-adenosyl-L-methionine, DL-normetanephrine hydrochloride, 3-amino propyl trimethoxysilane, sodium hydroxide, glutaric dialdehyde, tris hydrochloride and other chemicals unless otherwise stated were obtained from Sigma Chemical Co. (St. Louis, Mo., USA). An open tubular capillary (50 cm×100 μm ID) was purchased from Polymicron Technologies (ISA).

Procedure

An open tubular capillary (50 cm×100 μm ID) was attached to a vacuum through a 200 μl pipet tip and parafilm. The capillary was cleaned with 0.5 N NaOH by passing it via vacuum suction for 1 hour at room temperature The process was repeated with distilled deionized water for an additional 30 minutes. The water was removed by vacuum suction and the capillary was then placed in a GC 5890 oven at 95° C. for 1 hour.

A solution of APTS (3-amino propyl triethoxysilane) 10 parts and 90 parts water was passed through by vacuum suction at room temperature for 10 minutes and then dried at 95° C. for 30 minutes. This was repeated twice. Subsequently, a gluteraldehyde solution (1% v/v) in 50 mM PBS pH 7.0 was passed through the capillary for 1 hour at room temperature. A PNMT solution of 1 mg/mL (25 mM phosphate buffer, pH 8.4) was applied to the capillary with vacuum suction for 1 hour at room temperature. Both ends of the capillary were then placed in the remaining enzyme solution and incubated overnight.

The following day a solution of 0.5 M Tris-HCl buffer pH 7.5 was passed through the capillary for 30 minutes at room temperature followed by the 25 mM Phospate buffer solution pH 8.5 for 1 hr. The capillary was stored in the cold room with both ends immersed in the phosphate buffer solution.

Assay of PNMT Activity on Capillary

The activity of immobilized PNMT on the capillary was assayed as follows: A solution containing the substrate, normetanephrine (2.25 mM) and the cofactor, S-adenosyl-L-methionine (25 μM) was prepared. This solution (20 μl) was pumped through the capillary for 30 seconds at a flow rate of 0.1 mL/min, and incubated for 30 minutes. Subsequently, the capillary was connected to the PE-SCI-EX API-100 MS and was run in negative ion mode.

Results

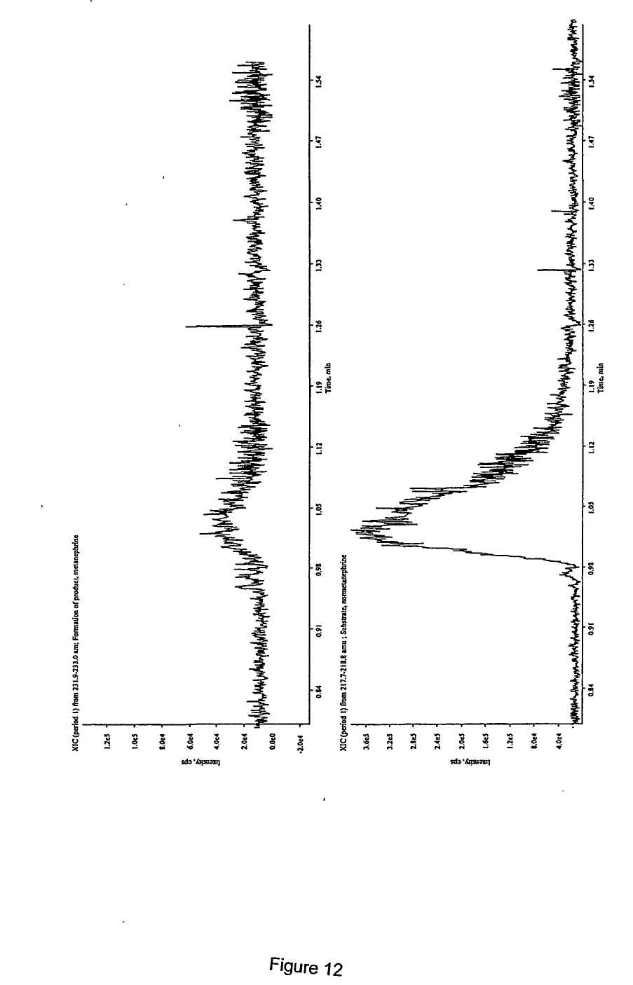

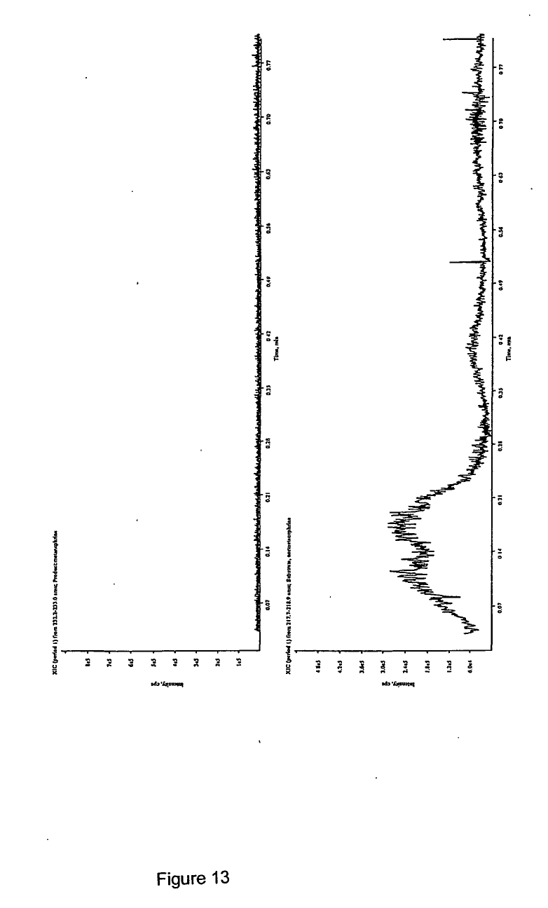

Single ion recordings were carried out for two molecular weights: 219.7 (Normetanephrine) and 233.7 (Metanephrine) in negative ion mode. The reaction mixture (substrate with cofactor) was analyzed. As shown in FIG. 1, the production of product, metanephrine, (FIG. 12) was seen at about 10% of the final concentration of substrate, normetanephrine (FIG. 13).

A control solution (substrate without cofactor) was also analyzed. In this case, no production of the product metanephrine was seen. These results confirmed the successful immobilization of active PNMT onto the surface of the open tubular capillary.

Discussion

The final two enzymes in the pathway, DBH and PNMT, display distinct activities and co-factor requirements. Thus, the multiple-IMER-HPLC system demonstrated the ease to connect vastly different enzymes in a single on-line system and the ability to deal with each enzyme individually if needed. Any one of the steps involved in the biosynthesis of catecholamines could assume a rate-limiting role depending upon pathological or pharmacologically induced situations. For instance, reserpine can transform DBH into a rate-limiting step by blocking the access of DA to its site for the conversion into NE. The IMERs can be used to investigate the potential of newly synthesized compounds that are believed to have reserpine-like effects. This would allow for the discovery of new therapeutic agents.

Complete characterization of immobilized dopamine β-hydroxylase was carried out and comparisons to the non-immobilized enzyme were made. Using immobilized artificial membrane(IAM) and glutaraldehyde-P (Glut-P) stationary phases to immobilize DBH allowed for the development of stationary phases that can be utilized to discover and characterize new drug candidates specific for membrane-bound and soluble forms of the enzyme. In vivo, most of DBH is largely recovered as a component of the vesicle membrane i.e. in its membrane-bound form, mDBH (Dixon et al., 1975; Edwards et al., 1980). Most studies that have investigated DBH activity and the development of potential inhibitors for various clinical conditions have been based upon the use of the soluble forms of the enzyme, sDBH. The development of the two IMER systems (DBH-Glut-P-IMER and DBH-IAM-IMER) allows for the selective screening of potential inhibitor candidates for the soluble and membrane-bound form of the enzyme respectively.

Although the present invention has been described hereinabove by way of preferred embodiments thereof, it can be modified, without departing from the spirit and nature of the subject invention as defined in the appended claims.

### LIST OF REFERENCES

References for Examples 1 and 2

Aunis, D., Bouclier, M., Pescheloche, M., Mandel, P. (1977). Properties of membrane-bound dopamine beta-hydroxylase in chromaffin granules from bovine adrenal medulla. J Neurochem 29: 439-47.

Bickerstaff, G. F. (1984). Applications of immobilized enzymes to fundamental studies on enzyme structure and function, in Topics in Enzyme and Fermentation Biotechnology, vol. 9, Ellis Horwood, Chichester, UK. pp. 162-201.

Bickerstaff, G. F. (1997). Immobilization of Enzymes and Cells, Humana Press, Totowa, N.J. pp. 140.

Boulton, A. A., Baker, G. B., Yu, P. H. (1990). Neuromethods: Neurotransmitter Enzymes, Humana Press Inc., Clifton, N.J. Pp. 117-143.

Cooper, J. R, Bloom, F. E., Roth, R. H. (1996). The Biochemical Basis of Neuropharmacology, 7^{th} ed., Oxford University Press. pp. 79-115.

Dixon, M., and Webb, E. C. (1979). Enzymes, 3^{rd}, Academic Press. pp. 60-130.

Friedman, S., and Kaufman, S. (1965). 3,4dihydroxyphenylethylamine beta-hydroxylase. Physical properties, copper content, and role of copper in the catalytic activity, J. Biol Chem., 240:4763-73.

Grunewald, G. L., Dahanukar, V. H., Ching, P., Criscione, K R. (1996). Effect of ring size or an additional heteroatom on the potency and selectivity of bicyclic benzylamine-type inhibitors of phenylethanolamine N-methyltransferase. J Med Chem. 39, 353946.

Helle, K B., Reed, R. Y, Phil, K. E. and Serck-Hanssen, G., Membrane dopamine beta-hydroxylase: a precursor for the soluble enzyme in the bovine adrenal medulla, Int. J. Biochem. 16 (1984) 641-50.

Johansson, K., Joensson-Pettersson, G., Gorton, L., Marko-Varga, G. and Csoregi, E., A reagentless amperometric biosensor for alcohol detection in column liquid chromatography based on co-immobilized peroxidase and alcohol oxidase in carbon paste, J. Biotechnol. 31 (1993) 301-16.

Lewis, E. J. and Asnani, L. P., Soluble and membrane-bound forms of dopamine beta-hydroxylase are encoded by the same mRNA, J. Biol. Chem., 267 (1992) 494-500.

Lowe, C. R., Hin, B. F., Cullen, D. C., Evans, S. E., Stephens, L. D., Maynard, P., Biosensors, J. Chromatogr. 510 (1990) 347-54.

Markoglou, N., and Wainer, I. W. Synthesis and Characterization of Immobilized DBH in Membrane Bound and Solubilized Formats. J Biochemical and Biophysical Methods 48 (2001) 61-75.

Marko-Varga, G., Johansson, K., Gorton, L., Enzyme-based biosensor as a selective detection unit in column liquid chromatography, J. Chromatogr. A., 660 (1994) 153-67.

Miras-Portugal, M. T., Aunis, D., Mandel, P., Human serum dopamine-beta-hydroxylase: purification, molecular weight, presence of sugars and kinetic properties, Biochimie, 57 (1975) 669-75.

Molinoff, P. B., Weinshilboum, R., and Axelrod, J., A sensitive enzymatic assay for dopamine β-hydroxylase, J. Pharmacol. Exp. Ther. 178 (1971) 425-31.

Mueller, S. A., Driscoll, W. J., Mueller, G. P., Captopril inhibits peptidylglycine-alpha-hydroxylating monooxygenase: implications for therapeutic effects, Pharmacology, 58 (1999) 270-80.

Nagatsu, T., Hidaka, H., Kuzuya, H., Takeya, K., Umezawa, H., Inhibition of dopamine β-hydroxylase by fusaric acid (5-butylpicolinic acid) in vitro and in vivo, Biochem. Pharmacol., 19 (1970) 3544.

Nagatsu, T. and Udenfriend, S. Photometric assay of dopamine-β-hydroxylase activity in human blood, Clin. Chem., 18 (1972) 980-83.

Nagatsu, T., Application of high-performance liquid chromatography to the study of biogenic amine-related enzymes, J Chromatogr., 566 (1991) 287-307.

Narayanan, S. R., Kakodkar, S. V., Crane, L. J., “Glutaraldehyde-P”, a stable, reactive aldehyde matrix for affinity chromatography, Anal Biochem., 188 (1990) 278-84.

Nordling, M., Elmgren, M., Stahlberg, J., Pettersson, G., Lindquist, S. E., A combined cellobiose oxidase/glucose oxidase biosensor for HPLC determination on-line of glucose and soluble cellodextrins., Anal. Biochem., 214 (1993) 389-96.

Palatini, P., Dabbeni-Sala, F., Finotti, P., inhibition of dopamine beta-hydroxylase by captopril, Biochem. Pharmacol., 36 (1989) 1011-13.

Pidgeon, C., Marcus, C., Alvarez, F., Applications of Enzyme Biotechnology, In T. O. Baldwin, and J. W. Kelly (Eds), New York, Plenum Press, 1992, pp. 201-220.

Tischer, W., Kasche, V., Immobilized enzymes: crystals or carriers?, Trends Biotecbnol., 17 (1999) 326-35.

Wong, D. L., and Bildstein, C. L., Subunit characterization and primary structure of bovine adrenal medullary dopamine beta-hydroxylase, Neuropsychopharmacology, 3 (1990) 115-28.

References for Example 3

Alebic-Kolbah, T., Felix, G. and Wainer, I. W. (1993). The on-line determination of the enantiomeric purity of enzymatically produced compounds. Chromatographia. 35, 264-268.

Alebic-Kolbah, T. and Wainer, I. W. (1993). Bioactive LC reactors for stereospecific synthesis with on-line enantioselective separations. Amer. Lab. October, 52R-52AA.

Alebic-Kolbah, T. and Wainer, I. W. (1993). Application of an enzyme-based stationary phase to the determination of enzyme kinetic constants and types of inhibition: A New High Performance Liquid Chromatographic Approach Utilizing An Immobilized Artificial Membrane Chromatographic Support. J. Chromatogr. A, 653, 122-129.

Alebic-Kolbah, T. and Wainer, I. W., (1993). Enzyme-based high performance liquid chromatographic phases as metabolic reactors: The immobilization of nonsolubilized rat liver microsomes on an immobilized artificial membrane high performance liquid chromatography support. J. Chromatogr. 646, 289-295.

Alebic-Kolbah, T. and Wainer, I. W. (1993). Microsomal immobilized enzyme reactor for on-line production of glucoronides in a high performance liquid chromatography column. Chromatographia, 37, 608-612.

Boulton, A. A., Baker, G. B., Yu, P. H. (1990). Neuromethods: Neurotransmitter Enzymes, Humana Press Inc., Clifton, N.J. Pp. 117-143.

Cahill A L, Eertmoed A L, Mangoura D, Perlman R L. (1996). Differential regulation of phenylethanolamine N-methyltransferase expression in two distinct subpopulations of bovine chromaffin cells. J Neurochem. 67, 1217-1224.

Chui W K, Wainer I W. (1992). Enzyme-based high-performance liquid chromatography supports as probes of enzyme activity and inhibition: the immobilization of trypsin and alpha-chymotrypsin on an immobilized artificial membrane high-performance liquid chromatography support. Anal Biochem. 201, 237-245.

Grunewald, G. L., Dahanukar, V. H., Ching, P., Criscione, K. R. (1996). Effect of ring size or an additional heteroatom on the potency and selectivity of bicyclic benzylamine-type inhibitors of phenylethanolamine N-methyltransferase. J Med Chem. 39, 353946.

Grunewald, G. L., Dahanukar, V. H., Jalluri, R. K., Criscion, K. R (1999). Synthesis and biochemical evaluation of 3-fluoromethyl-1,2,3,4-tetrahydroisoquinolines as selective inhibitors of phenylethanolamine N-methyltransferase versus the alpha(2)-Adrenoceptor. J Med Chem 42,3588-601.

Jadaud P, Thelohan S, Schonbaum G R, Wainer I W. (1989). The stereochemical resolution of enantiomeric free and derivatized amino acids using an HPLC chiral stationary phase based on immobilized alpha-chymotrypsin: chiral separation due to solute structure or enzyme activity. Chirality.1,38-44.

Johnson, D. V., Wahnon, D., Sotolongo, V. and Wainer I. W.(1997). On-line performance liquid chromatographic immobilized enzyme reactors for synthesis of stereochemically pure compounds. Chimica Oggi. 15, 104-107.

Molinoff P B, Landsberg L, Axelrod J. (1969). An enzymatic assay for octopamine and other beta-hydroxylated phenylethylamines. J Pharmacol Exp Ther. 170, 253-61.

Pasternyk, M., Ducharme, M. P., Descorps, V., Felix, G., Wainer, I. W. (1998). On-line deconjugation of chloramphenicol-beta-D-glucuronide on an immobilized beta-glucuronidase column Application to the direct analysis of urine samples. J. Chromatogr. A. 828, 135-140.

Pastemyk DiMarco, M., Felix, G., Descorps, V., Ducharme, M. P., Wainer, I. W. (1998). On-line deconjugation of glucoronides using an immobilized enzyme reactor based upon beta-glucuronidase. J. Chromatogr. B. 715, 379-386.

Saavedra, J. A., Palkovits, M., Brownstein, M. J., Axelrod, J. (1974) Localisation of phenylethanolamine N-methyltransferase in the rat brain nuclei. Nature. 248, 695-696.

Sotolongo, V., Johnson, D. V., Wahnon, D., Wainer, I. W. (1999) Immobilized horse liver alcohol dehydrogenase as an on-line high-performance liquid chromatographic enzyme reactor for stereoselective synthesis. Chirality. 11,39-45.

Thelohan, S., Jadaud, P., and Wainer, I. W. (1989).Immobilized enzyme as chromatographic phases for HPLC: The chromatography of free and derivatized amino acids on immobilized trypsin. Chromatographia. 28, 551-555.

Trocewicz, J., Kato, N., Oka, K., Nagatsu, T. (1982). Determination of phenylethanolamine N-methyltransferase activity in rat brain by high-performance liquid chromatography with fluorometric detection. J Chromatogr. 233, 328-33

Toomey, R. E., Homg, J. S., Hemrick-Luecke, S. K., Fuller, R. W.(1981). alpha 2-adrenoreceptor affinity of some inhibitors of norepinephrine N-methyltransferase. Life Sci. 24, 2467-72.

Vogel W H, Lewis L E, Boehme D H. (1976).Phenylethanolamine-N-methyltransferase activity in various areas of human brain, tissues and fluids. Brain Res 115,357-9

Zhang, X.-M. and Wainer, I. W. (1993). On-line determination of lipase activity and enantioselectivity using an immobilized enzyme reactor coupled to a chiral stationary phase. Tetrahedron. Lett., 34, 4731-4734.

References for Example 4

Markoglou, N. Wainer, I. W. Anal Biochem 288 (2001) 83.

Reference for Example 5

Yang, Q., Liu, X-Y., Miyake, J. (1998) Self-assembly and immobilization of liposomes in fused-silica capillary by avidin-biotin binding. Supramolecular Science 5, 769-772.

References for Introduction and Discussion

Alebic-Kolbah, T. and Wainer, I. W. (1993). Microsomal immobilized enzmye reactor for on-line production of glucoronides in high performance liquid chromatography column. Chromatographic, 37, 608-612.

Almas, B., Haavik, J., Flatmark, T. (1996) Characteization of a novel pterin intermediate formed in the catalytic cycle of tyrosine hydroxylase. Biochem. J., 319: 947-951.

Alaniz, R. C., Thomas, SA, Perez-Melgosa, M., Mueller, K., Farr, A. G., Palmiter, R. D., Wilson, C. B. (1999) Dopamine beta-hydroxylase deficiency impairs cellular immunity Proc Natl Acad Sci USA. 96:2274-2278.

Blakeborough, P., Louis, C. F., Turner, A. J. (1981) The structure and organization of dopamine-beta-hydroxylase in the chromaffin granule membrane. Biochim Biophys Acta. 669, 33-8.

Borchardt, R. T., Hubar, J. A., Wu, Y. S. (1976) Potential inhibitors of S-adenosyl-methionine-dependant methyltransferases. J. Med. Chem. 19,1094-1099.

Burke, W. J. Davis, J. W., Joh, T. H. (1983) The role of compartmetalization of epinephrine in the regulation of phenylethanolamine N-methyltransferase synthesis in rat adrenal medulla. Endocrinology 113:1102-1110.

Carmichael, S. W., Winkler, H.(1985) The adrenal chromaffin cell. Sci Am 253, 4049.

Connett, R. J., Kirsher, N. (1970) Purification and properties of bovine phenylethanolamine N-methyltransferase. J. Biol. Chem. 245, 329-334.

Cooper, J. R., Bloom, F. E., Roth, R. H. (1996) The Biochemical Basis of Neuropharmacology, 7^{th} edition, Oxford University Press, New York. pgs 226-269.

Dewolf, W. E., Chambers, P. A., Southan, C., Saunders, D., Kruse, L. I. (1989) Inactivation of dopamine beta-hydroxylase by β-ethynyltyramine: Kinetic characterization and covalent modification of an active site peptide. Biochemistry 28, 3833-3842.

Dixon, M. Webb, E. C. (1979) Enzymes, 3rd ed. Academic Press New York p 16-23.

Elenkov, I. J., Wilder, R. L., Chrousos, G. P., Vizi, E. S. (2000) The sympathetic nerve: An integrative interface between two supersystems the brain and immune system. Pharmacol Rev 52, 595-638.

Farrington, G. K., Kumar, A., Villafranca, J. J.(1990) Active site labeling of dopamine beta-hydroxylase by two mechanism-based inhibitors: 6-hydroxybenzofuran and phenylhydrazine. J Biol Chem. 265,1036-1040.

Friedman, J. I., Alder, D. N., Davis, K. L. (1999). The role of norepinephrine in the pathophysiology of cognitive disorders: potential applications to the treatment of cognitive dysfunction in schizophrenia and Alzheimer's disease. Biol Psychiatry 46, 1243-1252.

Grunewald, G. L., Carter, A. E., Sall, D. J., Monn, J. A. (1988) Conformational preference for the binding of biaryl substrates and inhibitors to the active site of phenylethanolamine N-methyltransferase. J Med Chem. 31, 60-65.

Grunewald, G. L., Dahanukar, V. H., Ching, P., Criscione, K. R. (1996) Effect of Ring Size or an Additional Heteroatom on the Potency and Selectivity of Bicyclic Benzylamine-Type Inhibitors of Phenylethanolamine N-Methyltransferase. J. Med. Chem. 39,3539-3546.

Helle, K. B., Reed, R. K., Pihl, K. E., Serck-Hanssen, G. (1984) Membrane dopamine beta-hydroxylase: a precursor for the soluble enzyme in the bovine adrenal medulla Int J Biochem. 16, 641-650.

Hoffman, R. M. (1984) Altered methinine metabolism, DNA methylation and oncogene expression in carcinogenesis. A review and synthesis. Biochim. Biophys Acta. 738,49-87.

Hoffman, A. S. (1990) Bioreactors in medicine. Biomater Artif Cells Artif Organs. 18:523-8.

Hurst, J. H., LeWitt, P. A., Burns, R. S., Foster, N. L., Lovenberg, W. (1985) CSF dopamine-beta-hydroxylase activity in Parkinson's disease. Neurology. 35:565-8.

Ishii, A., Kobayashi, K., Kiuchi, K., Nagatsu, T. (1991) Expression of two forms of human dopamine β-hydoxylase in COS cells. Neuroscience Letters 125, 25-28.

Joh, T. H. and Goldstein, M. (1973) Isolation and characterization of multiple forms of phenylethanolamine N-methyltransferase. Mol. Pharmacol. 9, 117-129.

Itoh, M. T., Hattori, A., Sumi, Y. (1997) Hydroxyindole-O-methyltransferase activity assay using high-performance liquid chromatography with flurometric detection: determination of melatonin enzymatically formed from N-acetylserotonin and SAM. J Chromatogr B 692, 217-221.

Jenne, D. F., Olsen, A. S., Zimmer, M. (1997) The human guanidinoacetate methyltransferase (GAMT) gene maps to syntenic region on 19p13.3, homologous to band C of mouse chromosome 10, but GAMT is not mutated in jittery mice. Biochem. Biophys Res Commun. 238,723-727.

Joh, T. H. and Goldstein, M. (1973) Isolation and characterization of multiple forms of phenylethanolamine N-methyltransferase. Mol. Pharmacol. 9, 117-129.

Kaneda, N., Ichinose, H., Kobayashi, K., Oka, K., Kishi, F., Nakazawa, A., Kurosawa, Y., Fujita, K., Nagatsu, T. (1988) Molecular cloning of cDNA and chromosomal assignment of the gene for human phenylethanolamine N-methyltransferase, the enzyme for epinephrine biosynthesis. J. Biol. Chem. 263, 7672-7677.

Katchalski-Katzir E. (1993) Immobilized enzymes: learning from past successes and failures. Trends Biotechnol. 11:471-8.

Katzung, B. G.(1995) Basic & Clinical Pharmacology 6^{th} ed. Appleton & Lange Norwalk, Conn., pgs. 9-31.

Kobayashi, K., Kurosawa, Y., Fujita, K., Nagatsu, T. (1989) Human dopamine beta-hydroxylase gene: two mRNA types having different 3′-terminal regions are produced through alternative polyadenylation. Nucleic Acids Res. 17, 1089-1102.

Kobayashi, K., Sasaoka, T., Morita, S., Nagatsu, I., Iguchi, A., Kurosawa, Y., Fujita, K., Nomura, T., Kimura, M., Katsuki, M. (1992) Genetic alteration of catecholamine specificity in transgenic mice. Proc Natl Acad Sci 89, 1631-1635.

Lamouroux, A., Vigny, A., Faucon Biguet, N., Darmon, M. C., Franck, R., Henry, J. P., Mallet, J. (1987) The primary structure of human dopamine beta-hydroxylase: insights into the relationship between the soluble and the mebrane-bound forms of the enzyme. EMBO J 6, 3931, 1987.

Liang J F, Li Y T, Yang V C. (2000) Biomedical application of immobilized enzymes. J Pharm Sci. 89:979-90. Review.

Ledbetter, F. H., Kirshner, N. (1981) Quantitative correlation between secretion and cellular content of catecholamines and dopamine-beta-hydroxylase in cultures of adrenal medulla cells. Biochem Pharmacol. 30, 3246-3249.

Lee, M., Nohta, H., Ohkura, Y., Yoo, B. (1985) Determination of phenylethanolamine N-methyltransferase by high performance liquid chromatography with fluorescence detection. J. Chromatogr. 348, 407415.

Liposits, Z., Phelix, C., Paull, W. K. (1986). Electron microscopic analysis of tyrosine hydroxylase, dopamine beta-hydroxylase and phenylethanolamine N-methyltransferase immunoreactive innervation of the hypothalamic paraventricular nucleus in the rat. Histochemistry. 84, 105-120.

Lough, W. J., Wainer, I. W., (1996) High Performance Liquid Chromatogrpahy: Fundamental Principles and Practise, Blackie Academic & Professional, Chapman & Hall, UK.

McMahon, A., Geertman, R., Sabban, E. L. Rat dopamine beta-hydroxylase: molecular cloning and characterization of the cDNA and regulation of the mRNA by reserpine. J Neurosci Res. 25, 395404.

Molinoff, P. B., Landsberg, L., Axelrod, J. (1969) An enzymatic assay for octopamine and other beta-hydroxylated phenylethylamines. The Journal of Pharmacology and Experimental Therapeutics. 170, 252-261.

Molinoff, P. B., Axelrod, J. (1971) Biochemistry of catecholamines. Annu Rev Biochem. 40, 465-500.

Nagatsu, T., Levitt, M., Udenfriend, S. (1964) Conversion of L-tyrosine to 3,4-dihydroxyphenylalanine by cell-free prepratations of brain and sympathetically innervated tissues. Biochem Biophys Res Commun. 14, 543-549.

Nagatsu, T., Kuzya, H., Hidaka, H.(1967) Inhibition of dopamine beta-hydroxylase by sulfhydryl compounds and the nature of the natural inhibitors. Biochimica et Biophysica Acta 139, 319-327.

Nagatsu, T., Levitt, M., Udenfriend, S. (1964) Conversion of L-tyrosine to 3,4-dihydroxyphenylalanine by cell-free prepratations of brain and sympathetically innervated tissues. Biochem Biophys Res Commun. 14, 543-549.

Nagatsu, T., Kuzya, H., Hidaka, H.(1967) Inhibition of dopamine beta-hydroxylase by sulffiydryl compounds and the nature of the natural inhibitors. Biochimica et Biophysica Acta 139, 319-327.

Nagatsu, T. Kobayashi, K., Ichinose, H. (1995) Alterations in catecholamine system enzymes. Ann N Y Acad Sci. 771, 159-165.

Nagatsu, T., Stjarne, L. (1998) Catecholamine synthesis and release. Advances in Pharmacology. 42, 1-13.

Park, D. H., Baetge, E. E., Kaplan, B. B., Albert, V. R., Reis, D. J., Joh, T. H. (1982) Different forms of adrenal phenylethanolamine N-methyltransferase species-specific posttranslational modification. J Neurochem. 38, 410-414.

Park, D. H., Ehrlinch, M., Evinger, M. J., Joh, T. H. (1986) Strain differences in distribution of phenylethanolamine N-methyltransferase activity from rat brain and adrenal gland. Brain Res. 372, 185-188.

Pendleton, R. G. and Snow, I. B. (1973) The Binding Order of Substrates to Phenylethanolamine N-Methyltransferase. Mol. Pharmacol. 9, 718-725.

Reis, D. J., Morrison, S., Ruggiero, D. A. (1988) The C1 area of the brainstem in tonic and reflex control of blood pressure. Hypertension. 11: 18-113.

Robertson, D., Haile, V., Perry, S. E., Robertson, R. M., Phillips, J. A. Biaggioni, I. (1991) Dopamine beta-hydroxylase deficiency-a genetic disorder of cardiovascular regulation, Hypertension 18,1-8.

Robertson, J. G., Desai, P. R., Kumar, A., Farrington, G. K., Fitzpatrick, P. F., villafranca, J. J. (1990) Primary amino acid sequence of bovine dopamine beta-hydroxylase. J Biol Chem 265, 1029-1035.

Robertson, J. G., Adams, G. W., Medzihradszky, K. F., Burlingame, A. L., Villafranca, J. J. (1994) Complete assignment of disulfide bonds in bovine dopamine beta-hydroxylase. Biochemistry. 33, 11563-11575.

Saavedra, J. M. (1979) Adrenaline levels in brain stem nuclei and effects of a PNMT inhibitor on spontaneously hypertensive rats. Brain Res. 166: 283-292.

Sabban, E. L., Greene, L. A., Goldstein, M. (1983) Mechanism of biosynthesis of soluble and membrane-bound forms of dopamine beta-hydroxylase in PC12 pheochromocytoma cells. J Biol Chem. 258, 7812-7818.

Sall, D. J., Grunewald, G. K. (1987) Inhibition of phenylethanolamine N-methyltransferase (PNMT) by aromatic hydroxy-substituted 1,2,3,4-tetrahydroisoquinolines: further studies on the hydrophilic pocket of the aromatic ring binding region of the active site. J Med Chem. 30, 2208-2216.

Schulser-van Hees, M. T., Beijersbergen van Henegouwen, G. M. (1980). Determination of catecholamines and O-methylated metabolites by reversed-phase high-performance liquid chromatography with fluorometric detection and its application to enzyme kinetics. J. chromatogr. 196, 101-108.

Tani, S., Muto, N. (1982) Effects of pentagastrin, histamine, carbamylcholine and catecholamines on gastric secretion, motility and emptying in the rat. Biochem Pharmacol 31, 3475-3481.

Thomas, S. A., Matsumoto, A. M., Palmiter, R. D. (1995) Noradrenaline is essential for mouse fetal development. Nature. 374:643-646.

Thomas, S. A. and Palmiter, R. D. (1997) Disruption of the dopamine beta-hydroxylase gene in mice suggests roles for norepinephrine in motor function, learning, and memory. Behav Neurosci. 111:579-89.

Thomas, S. A. and Palmiter, R. D. (1998) Examining adrenergic roles in development, physiology, and behavior through targeted disruption of the mouse dopamine beta-hydroxylase gene. Adv Pharmacol. 42:57-60.

Toomey, R. E., Horng, J. S., Hemrick-Leucke, S. K., Fuller, R. W. (1981) α₂-Adrenoceptor Affinity of Some Inhibitors of Norepinephrine N-Methyltransferase. Life Sci. 29: 2467-2472.

Turner, A. P. F., Karube, I., Wilson, G. S. (1987) Biosensors: Fundamentals and Applications. Oxford University Press, Oxford. pgs. 770-775.

Voltattorni, C. B., Minelli, A., Dominici, P. (1983) interaction of aromatic amino acids in D and L forms with 3,4dihydroxyphenylalanine decarboxylase from pig kidney. Biochemistry 22,2249-2254.

Wang, N., Southan, C., DeWolf, W. E. Jr., Wells, T. N., Kruse, L. I., Leatherbarrow, R. J. (1990) Bovine dopamine beta-hydroxylase, primary structure determined by cDNA cloning and amino acid sequencing. Biochemistry. Jul. 10, 1990;29(27):6466-74.

Wimalasena K, Wimalasena D S. (1991)Continuous spectrophotometric assays for dopamine beta-monooxygenase based on two novel electron donors: N,N-dimethyl-1,4-phenylenediamine and 2-aminoascorbic acid. Anal Biochem. 197,:353-61.

Winalasena, K. and Alliston, K. R. (1999) Mode of substrate interaction and energetics of carbon-oxygen bond formation of the dopamine beta-monooxygenase reaction. Biochemistry 38, 14916-14926.

Wong, D. L. and Bildstein, C. L. (1990) Subunit characterization and primary structure of bovine adrenal medullary dopamine beta-hydroxylase. Neuropsychopharmacology 3, 115-128.

What is claimed is:

1. Use of multiple immobilized enzyme reactors (IMERs) in a liquid chromatographic system to perform enzymatic transformations or compound syntheses.

2. A use as defined in claim 1, wherein said system utilizes a separate MER for each enzyme selected to perform said enzymatic transformations or compound syntheses.

3. A use as defined in claim 1 or 2, wherein said system utilizes on-line purification and identification of the products after each enzymatic transformation.

4. A use as defined in claim 3, wherein said system further permits the recycling of unreacted starting materials to optimize their use.

5. A use as defined in any one of claims 1 to 4, wherein said system can be used for designed synthetic pathways or to mimic natural biosynthetic pathways.

6. A use as defined in any one of claims 1 to 4, wherein said system can be used to screen chemical entities for the discovery or characterization of new drug candidates.

7. A use as defined in claim 6, wherein each IMER in said system can be used separately for the high throughput screening of chemical entities for the discovery or characterization of new drug candidates.

8. A system comprising multiple immobilized enzyme reactors (IMERs) for performing enzymatic transformations, compound syntheses, high throughput screening, drug discovery or drug characterization.

9. A system as defined in claim 8, wherein individual IMERs are utilized for each enzyme selected for performing said enzymatic transformations, compound syntheses, high throughput screening, drug discovery or drug characterization.

10. A system as defined in claim 8, wherein said IMERs are constructed by covalent immobilization or hydrophobic entrapment on silica-based chromatographic supports.

11. A system as defined in claim 9, wherein said IMERs are open tubular columns containing immobilized enzymes.

12. A system as defined in claim 10 or 11, wherein said system is coupled to a mass spectrometer or any other detection device.

13. Use of a sytem as defined in any one of claims 8-12 to identify new drugs.

14. Use of said individual IMERs of claim 9 or 11 to identify new drugs.

15. A system as defined in any one of claims 8-12 for the enzymatic transformation of tyrosine to epinephrine.

16. A system as defined in claim 15, wherein said IMERs consist of tyrosine hydroxylase, dopa decarboxylase, dopamine β-hydroxylase and phenylethanolamine N-methyltransferase covalently immobilized and hydrophobically entrapped on silica-based liquid chromatographic supports.

17. A system as defined in claim 8 or 9 for the production of a modular liquid chromatographic system for the four-step synthesis of epinephrine from tyrosine.

18. A system as defined in claim 17, further comprising on-line separation and purification of products and substrates.
